SEQUENCE LISTING (36 sequences)

SEQ ID NO: 1 - DNA, 514 bp, Artificial Sequence
  CsVMV Promoter
caaacttacaaatttctctgaagttgtatcctcagtacttcaaagaaaatagcttacaccaaattttttcttgttttcacaaatgccgaacttggttccttatataggaaaactcaagggcaaaaatgacacggaaaaatataaaaggataagtagtgggggataagattcctttgtgataaggttactttccgcccttacattttccaccttacatgtgtcctctatgtctctttcacaatcaccgaccttatcttcttcttttcattgttgtcgtcagtgcttacgtcttcaagattcttttcttcgcctggttcttctttttcaatttctatgtattcttcttcgtattctggcagtataggatcttgtatctgtacattcttcatttttgaacataggttgaatatgtgccgcatattgatctgcttcttgctgagttcacataatacttccatagtttttcccgtaaacattggattcttgatgctacatcttggataattaccttctg

SEQ ID NO: 2 - DNA, 546 bp, Artificial Sequence
  Dahlia MV Promoter
caatcctcctcaggaaatgaaggattcaggagatcatctctatcaacttgctcaagtaaggacaaacgggttcacccggatcctccagaagacccagtctatcaacggagaaacaaagataaaaatcaattactcacatgaaagagtattgatcacgagtcactatggagcgacaatctccagacaggatgtcagcatcttatcttcctttgaagaaagcatcatcaataacgatgtaatggtggggacatccactaagttattgctctgcaaacagctcaaaaagctactggccgacaatcataattgctcggcatgtcaggtggggcctccactagcaataatacaagctttacagcttgcagtgactcatcctccaataatgaggaaaaagacgtcagcagtgacgaacaagggcctgaagacttgcctatataatggcattcacccctcagttgaagagcatcaggagtttcagcatagaaactttctctttaacaaatctatcttttctttaaagcatgtgtgagtagaaacccatatagg

SEQ ID NO: 3 - DNA, 241 bp, Artificial Sequence
  PCLSV Promoter
tgagccaatcaaagaggagtgatgtagacctaaagcaataatggagccatgacgtaagggcttacgccattacgaaataattaaaggctgatgtgacctgtcggtctatcagaacctttactttttatatttggcgtgtatttttaaatttccacggcaatgacgatgtgacctgtggatccgctttgcctataaataagttttagtttgtattgatcgacacgatcgagaagacacggcc

SEQ ID NO: 4 - DNA, 500 bp, Artificial Sequence
  HLVH12 Promoter
tggtagactatgaaacactagtctactcaaagaacttgaagaagacgactcaggaagacaggagcgtcatcaacaagtttcagcaaaagctgattagtggaaaaatccttggattccactctccagcaatctgccagcacataaaggtgacagcagaaaaagaagattgtgactaccactgcaatcagtgcgaatcttcaaaaggaaaggctatcgtttgcgataagcctgccgacagtggtccagccgacaatggagggccccagacacgtgaagacgcgtctgccgacagtgggtctaggacaacagacaccacgcactcagcaagtggatgaaataattcatctgctgacgtaagggatgacgatcaatcccactatcccaagacccttcacttctatataagtgaagttgcttcatttggagaaggcatctcgaaatctcaacacaactcgagctctcccttctctcttctttatctctctaaatgtgtgagtaga

SEQ ID NO: 5 - DNA, 1336 bp, Artificial Sequence
  Watermelon AGPase Fruit Promoter
gttaatatgtaaaatcgataatgcaaaaatattttaactatttattttgagatgaaataaagtaggagcgatcttaatcttccaattgtaaaaacatacgtacacatctaaattttatacttaaggaggtgtttgggcgtgactttaaatgggtggggtaaactatcatagctcactccatgtttaagaatgtagttatagtaattggtgtttccaactattataattcacaattaactaccgtattacttgttacaatatttactattttccacctatcttctcttcccctaattgttatagtgtttattattacttagactaaaatagtatgcaccttatacatgcagaagtagaatagtaatatagtttaaaactacattggtccagtatcaagaagatatcttaattttatttaggaatttaataattgggtgtggttttgtaaacataaaaataaagaaagctataaacagaggtcctaggatgcagattggttacagaattcagtgccccaggaagtagcaccaattttcatcatttatttaaaaagtatattttgaagaaatattattgtgaaatagtttttgaatttgttgcgtgcttggtggaagtgggcaacacaaatacatgggtgcccatccaagtattgccttgtttgtttgtttgtatgcggcgtagaaatagcggatgatcgttggaaattgagttttgtaggaatagcaaaaaaagaaagaaagaaatatgaagaagatgaagatgtaaatgaggaagaagaagaaccatttgctgacatgaatgaacctttcccactttcttgttttttactataaatcaatcctcgtgaatgaaaacgccttacattcacatgcccattaagcattaatcccctttctccaccgccttcttcatcatcactcacatttgtcactctcttttcctctttctctcttcgtcttcttcccccatttccaacgcttctacctttgattcgtttcccctttgtaagttttcgatttcttctgctttcctttctgggatttcttatttgcatcatttacttttctgggtgtctattattttattgtattagagctttgtcagatgatttcttgtatttgtttagctaccccttttgcttttttctgttcttggtgtgatctgtactctatatggttgcttggattggggcttttgctttttcttattgggatttgagctgggggtggggctattagattagattgtaatttgtgccatttctgatgcatttttttttttttccaagggagagagttaactgatttcttgaatttgttatcgctagtgagataac

SEQ ID NO: 6 - DNA, 486 bp, Artificial Sequence
  AtHSP18.2 Terminator
gaagatgaagatgaaatatttggtgtgtcaaataaaaagcttgtgtgcttaagtttgtgtttttttcttggcttgttgtgttatgaatttgtggctttttctaatattaaatgaatgtaagatctcattataatgaataaacaaatgtttctataatccattgtgaatgttttgttggatctcttctgcagcatataactactgtatgtgctatggtatggactatggaatatgattaaagataagatgggctcatagagtaaaacgaggcgagggacctataaacctcccttcatcatgctatttcatgatctattttataaaataaagatgtagaaaaaagtaagcgtaataaccgcaaaacaaatgatttaaaacatggcacataatgaggagattaagttcggtttacgtttattttagtactaattgtaacgtgagactacgtatcgggaatcgcctaattaaagcattaatgcgaacctg

SEQ ID NO: 7 - protein, 53 aa, Artificial Sequence
  Non-Epitope Tagged Versions, des-pyrE-Brazzein
DKCKKVYENYPVSKCQLANQCNYDCKLDKHARSGECFYDEKRNLQCICDYCEY

SEQ ID NO: 8 - DNA, 159 bp, Artificial Sequence
  Non-Epitope Tagged Versions, des-pyrE-Brazzein
atattcacaataatcacaaatacattgaagatttctcttttcatcataaaaacattctccagatctagcatgcttatcaagcttacaatcataattacattgattagcaagttgacacttagaaacaggataattttcataaaccttcttacacttatc

SEQ ID NO: 9 - protein, 76 aa, Artificial Sequence
  Non-Epitope Tagged Versions, BAAS-des-pyrE-Brazzein
MGKKSHICCFSLLLLLFAGLASGDKCKKVYENYPVSKCQLANQCNYDCKLDKHARSGECFYDEKRNLQCICDYCEY

SEQ ID NO: 10 - DNA, 228 bp, Artificial Sequence
  Non-Epitope Tagged Versions, BAAS-des-pyrE-Brazzein
atattcacaataatcacaaatacattgaagatttctcttttcatcataaaaacattctccagatctagcatgcttatcaagcttacaatcataattacattgattagcaagttgacacttagaaacaggataattttcataaaccttcttacacttatctccagaagcaagtccagcaaaaagaagaagaagaagagaaaaacaacaaatatgagacttctttcccat

SEQ ID NO: 11 - protein, 73 aa, Artificial Sequence
  Non-Epitope Tagged Versions, PR1a-des-pyrE-Brazzein
MKSSIFVACFITFIIFHSSQDKCKKVYENYPVSKCQLANQCNYDCKLDKHARSGECFYDEKRNLQCICDYCEY

SEQ ID NO: 12 - DNA, 222 bp, Artificial Sequence
  Non-Epitope Tagged Versions, PR1a-des-pyrE-Brazzein
atattcacaataatcacaaatacattgaagatttctcttttcatcataaaaacattctccagatctagcatgcttatcaagcttacaatcataattacattgattagcaagttgacacttagaaacaggataattttcataaaccttcttacacttatcagcttgagaagaatgaaaaataataaaagtaataaaacaagcaacaaaaatagaagacttcat

SEQ ID NO: 13 - protein, 61 aa, Artificial Sequence
  Codon Optimized, des-pyrE-Brazzein-FLAG
DKCKKVYENYPVSKCQLANQCNYDCKLDKHARSGECFYDEKRNLQCICDYCEYDYKDDDDK

SEQ ID NO: 14 - DNA, 183 bp, Artificial Sequence
  Codon Optimized, des-pyrE-Brazzein-FLAG WT
gataagtgtaagaaggtttatgaaaattatcctgtttctaagtgtcaacttgctaatcaatgtaattatgattgtaagcttgataagcatgctagatctggagaatgtttttatgatgaaaagagaaatcttcaatgtatttgtgattattgtgaatatgactacaaagatgacgatgataag

SEQ ID NO: 15 - DNA, 183 bp, Artificial Sequence
  Codon Optimized, des-pyrE-Brazzein-FLAG Maize
gacaagtgtaaaaaggtgtatgagaactatcctgtttcaaaatgtcagcttgctaaccaatgtaattacgattgtaaactggataagcacgctaggtctggtgaatgcttttatgacgaaaaacgcaaccttcaatgtatatgcgattattgcgaatatgactataaggacgatgatgataag

SEQ ID NO: 16 - DNA, 183 bp, Artificial Sequence
  Codon Optimized, des-pyrE-Brazzein-FLAG Canola
gataaatgcaaaaaggtgtacgaaaactacccagtgtcaaagtgccaactggcgaaccaatgcaactacgactgtaaacttgacaagcacgcccgtagcggggagtgcttttatgatgagaagagaaacctccagtgcatctgtgattattgcgagtacgattacaaggacgacgacgataag

SEQ ID NO: 17 - DNA, 183 bp, Artificial Sequence
  Codon Optimized, des-pyrE-Brazzein-FLAG Tobacco
gataaatgcaagaaagtgtacgagaattatccggtaagtaaatgtcaactcgccaatcaatgcaactacgactgtaaattagataaacacgctagaagcggagaatgcttctacgacgagaaaaggaatttgcaatgtatctgtgactattgcgagtacgactacaaagatgacgatgataaa

SEQ ID NO: 18 - DNA, 183 bp, Artificial Sequence
  Codon Optimized, des-pyrE-Brazzein-FLAG Tomato
gacaaatgtaaaaaggtttatgaaaactatcccgttagtaagtgccagcttgctaatcaatgtaactatgactgcaagttggacaagcatgctaggtcaggtgagtgtttttacgacgaaaagcgaaatctacagtgtatttgcgactactgcgagtacgactacaaagatgacgacgataaa

SEQ ID NO: 19 - DNA, 183 bp, Artificial Sequence
  Codon Optimized, des-pyrE-Brazzein-FLAG Potato
gacaagtgcaaaaaggtttacgagaactatccagtgagtaagtgtcaattagcaaaccaatgcaattacgattgcaaactagataaacatgctcgaagtggggagtgtttctacgatgaaaagcgaaacctacagtgcatctgcgactattgcgagtacgactacaaggacgacgacgataag

SEQ ID NO: 20 - protein, 84 aa, Artificial Sequence
  Epitope Tagged Versions, BAAS-des-pyrE-Brazzein-FLAG
MGKKSHICCFSLLLLLFAGLASGDKCKKVYENYPVSKCQLANQCNYDCKLDKHARSGECFYDEKRNLQCICDYCEYDYKDDDDK

SEQ ID NO: 21 - DNA, 252 bp, Artificial Sequence
  Epitope Tagged Versions, BAAS-des-pyrE-Brazzein-FLAG
atgggaaagaagtctcatatttgttgtttttctcttcttcttcttctttttgctggacttgcttctggagataagtgtaagaaggtttatgaaaattatcctgtttctaagtgtcaacttgctaatcaatgtaattatgattgtaagcttgataagcatgctagatctggagaatgtttttatgatgaaaagagaaatcttcaatgtatttgtgattattgtgaatatgactacaaagatgacgatgataag

SEQ ID NO: 22 - protein, 81 aa, Artificial Sequence
  Epitope Tagged Versions, PR1a-des-pyrE-Brazzein-FLAG
MKSSIFVACFITFIIFHSSQDKCKKVYENYPVSKCQLANQCNYDCKLDKHARSGECFYDEKRNLQCICDYCEYDYKDDDDK

SEQ ID NO: 23 - DNA, 186 bp, Artificial Sequence
  Epitope Tagged Versions, PR1a-des-pyrE-Brazzein-FLAG
atgaagtcttctatttttgttgcttgttttattacttttattatttttcattcttctcaagctgataagtgtaagaaggtttatgaaaattatcctgtttctaagtgtcaactttatgatgaaaagagaaatcttcaatgtatttgtgattattgtgaatatgactacaaagatgacgatgataag

SEQ ID NO: 24 - protein, 84 aa, Artificial Sequence
  Epitope Tagged Versions, CHIA-des-pyrE-Brazzein-FLAG
MSSTKLISLIVSITFFLTLQCSMADKCKKVYENYPVSKCQLANQCNYDCKLDKHARSGECFYDEKRNLQCICDYCEYDYKDDDD

SEQ ID NO: 25 - DNA, 255 bp, Artificial Sequence
  Epitope Tagged Versions, CHIA-des-pyrE-Brazzein-FLAG
atgtcttctactaagcttatttctcttattgtttctattactttttttcttactcttcaatgttctatggctgataagtgtaagaaggtttatgaaaattatcctgtttctaagtgtcaacttgctaatcaatgtaattatgattgtaagcttgataagcatgctagatctggagaatgtttttatgatgaaaagagaaatcttcaatgtatttgtgattattgtgaatatgactacaaagatgacgatgataag

SEQ ID NO: 26 - protein, 83 aa, Artificial Sequence
  Epitope Tagged Versions, BP80-des-pyrE-Brazzein-FLAG
MKCWRLSAILFLGFMLTSLSTADKCKKVYENYPVSKCQLANQCNYDCKLDKHARSGECFYDEKRNLQCICDYCEYDYKDDDDK

SEQ ID NO: 27 - DNA, 249 bp, Artificial Sequence
  Epitope Tagged Versions, BP80-des-pyrE-Brazzein-FLAG
atgaagtgttggagactttctgctattctttttcttggatttatgcttacttctctttctactgctgataagtgtaagaaggtttatgaaaattatcctgtttctaagtgtcaacttgctaatcaatgtaattatgattgtaagcttgataagcatgctagatctggagaatgtttttatgatgaaaagagaaatcttcaatgtatttgtgattattgtgaatatgactacaaagatgacgatgataag

SEQ ID NO: 28 - protein, 93 aa, Artificial Sequence
  Epitope Tagged Versions, S25-des-pyrE-Brazzein-FLAG
MANKLFLVCATFALCFLLTNADKCKKVYENYPVSKCQLANQCNYDCKLDKHARSGECFYDEKRNLQCICDYCEYDYKDDDDKCEYDYKDDDDK

SEQ ID NO: 29 - DNA, 279 bp, Artificial Sequence
  Epitope Tagged Versions, S25-des-pyrE-Brazzein-FLAG
atggctaataagctttttcttgtttgtgctacttttgctctttgttttcttcttactaatgctgataagtgtaagaaggtttatgaaaattatcctgtttctaagtgtcaacttgctaatcaatgtaattatgattgtaagcttgataagcatgctagatctggagaatgtttttatgatgaaaagagaaatcttcaatgtatttgtgattattgtgaatatgactacaaagatgacgatgataagtgtgaatatgactacaaagatgacgatgataag

SEQ ID NO: 30 - DNA, 159 bp, Artificial Sequence
  Gene encoding brazzein, des-pyrE-Brazzein
gataagtgtaagaaggtttatgaaaattatcctgtttctaagtgtcaacttgctaatcaatgtaattatgattgtaagcttgataagcatgctagatctggagaatgtttttatgatgaaaagagaaatcttcaatgtatttgtgattattgtgaatat

SEQ ID NO: 31 - DNA, 23 bp, Artificial Sequence
  Gene encoding FLAG
actacaaagatgacgatgataag

SEQ ID NO: 32 - DNA, 69 bp, Artificial Sequence
  Gene encoding BAAS
atgggaaagaagtctcatatttgttgtttttctcttcttcttcttctttttgctggacttgcttctgga

SEQ ID NO: 33 - DNA, 63 bp, Artificial Sequence
  Gene encoding signal peptide PR1
atgaagtcttctatttttgttgcttgttttattacttttattatttttcattcttctcaagct

SEQ ID NO: 34 - DNA, 72 bp, Artificial Sequence
  Gene encoding signal peptide CHIA
atgtcttctactaagcttatttctcttattgtttctattactttttttcttactcttcaatgttctatggct

SEQ ID NO: 35 - DNA, 66 bp, Artificial Sequence
  Gene encoding signal peptide BP80
atgaagtgttggagactttctgctattctttttcttggatttatgcttacttctctttctactgct

SEQ ID NO: 36 - DNA, 63 bp, Artificial Sequence
  Gene encoding signal peptide S25
atggctaataagctttttcttgtttgtgctacttttgctctttgttttcttcttactaatgct

(19) United States
(12) Patent Application Publication
(10) Pub. No.: US 2024/0262874 A1
(43) Pub. Date: Aug. 8, 2024

(54) NOVEL BRAZZEIN PRODUCTION SYSTEM AND METHODS

(71) Applicant: The Coca-Cola Company, Atlanta, GA (US)

(72) Inventors: Christopher P. MERCOGLIANO, Atlanta, GA (US); Indra PRAKASH, Atlanta, GA (US); Alec HAYES, Durham, NC (US); Fayaz KHAZI, Durham, NC (US); Tengfang HUANG, Durham, NC (US)

(21) Appl. No.: 18/565,025

(22) PCT Filed: May 27, 2022
(86) PCT No.: PCT/US2022/031322

§ 371 Date: Nov. 28, 2023

Related U.S. Application Data
(60) Provisional application No. 63/194,552, filed on May 28, 2021.

(51) Int. Cl. C07K 14/43; C12N 15/82

(52) U.S. Cl. CPC C07K 14/43; C12N 15/8243

(57) ABSTRACT

The present disclosure presents a solution to producing a sweet protein or a variant thereof having low or no calorie. By using recombinant gene and plant transformation techniques, non-native genes encoding a sweet protein are included in the propagatable genome of a plant, thereby forming a genetically modified plant, wherein the plant by the native genome thereof prior to modification may not produce the sweet protein naturally. Such genetically modified plant and a progeny thereof are enabled to produce non-native sweet protein and/or a variant thereof. Sweeteners, compositions, and consumables derived from the genetically modified plant are also provided.

This application is being filed on May 27, 2022, as a PCT International Patent application and claims the benefit of and priority to U.S. Provisional patent application Ser. No. 63/194,552, filed May 28, 2021, the entire disclosure of which is incorporated by reference herein in its entirety.

Pursuant to 37 C.F.R. § 1.821(c) or (e), a file containing an ASCII text version of the Sequence Listing has been submitted concomitant with this application, the contents of which are hereby incorporated by reference.

### INTRODUCTION

With the increased awareness of healthy diets and potential risks of obesity and diabetes in global countries, low or non-calorie sweetener alternatives to traditional high calorie sweeteners are becoming increasingly important to the food and beverage business as well as other industries. Some consumers are also interested in using sweeteners that can be found in nature as substitutes to artificial sweeteners or high calorie sweeteners comprising sucrose, fructose, and glucose Like some artificial sweeteners, natural sweeteners can provide a greater sweetening effect than comparable amounts of caloric sweeteners; thus, smaller amounts of these alternatives are required to achieve sweetness comparable to that of sugar. However, some of these sweeteners found in nature can be expensive to produce and/or possess unfavorable taste characteristics and/or off-tastes, including but not limited to sweetness linger, delayed sweetness onset, negative mouth feels, bitter, metallic, cooling, astringent, and licorice-like tastes.

Sweet proteins as alternative sweeteners have received great attention. Until now, few sweet proteins have been isolated: thaumatin, monellin, mabinlin, brazzein, egg white lysozime, and neoculin (Masuda, 2005), and pentadin (van der Wel, 1989). These proteins are several thousand or hundred times sweeter than sucrose on a weight basis (Kant, 2005).

Brazzein is a sweet protein which can be extracted from the fruit of the West African climbing plant Pentadiplandra brazzeana Baillon (WO9531547). It has been characterized as a monomer protein having a 3-dimensional structure with four evenly spaced di-sulfide bonds. Three forms of the protein are known to exist in nature differing only at the N-terminal amino acid residue. One corresponds to the 54-amino acid translation product containing a glutamine at its N-terminus. This form has been shown to be short lived as the N-terminal glutamine undergoes natural conversion to pyroglutamate, resulting in the second form (Ming et al., 1994). The loss of the N-terminal glutamine or pyroglutamate yields the 53-amino acid form which has been reported to be twice as sweet as the form having an N-terminal pyroglutamate (Izawa er al., 1996).

Among the sweet tasting proteins isolated, brazzein seems to be the most promising one (Faus, 2000). In fact, its sweet perception is more similar to sucrose than that of the other sweet proteins (Pfeiffer et al., 2000). Furthermore, it possesses better pH and thermal stabilities in comparison to the other sweet-tasting proteins. It was demonstrated that its sweetening power does not diminish after incubation at 98° C. for 4 hours; moreover, it is stable over a broad pH range (2.5 to 8). It was also demonstrated that brazzein is very soluble in water (>50 mg/ml) (Ming et al., 1994). These proprieties made the protein suitable for many industrial food manufacturing processes as a low-calorie sweetener.

Brazzein can be chemically synthesized (Izawa et al., 1996), which is useful for its production in small scale for structure-function studies, but not suitable for large scale commercial production. Additionally, chemical synthesis is expensive.

A method for the recombinant expression of brazzein in Escherichia coli has been reported (Assadi-Porter et al., 2000). However, even in a bacterial system is ideal for its ease of rapid genetic manipulation as well as isotopic labeling for structural investigation, it is unsuitable for the production of protein for human consumption. The biosynthetic production of brazzein has also been disclosed in P. pastoris yeast (Carlson, US20100112639), filamentous fungus (Vind, U.S. Pat. No. 9,273,320), maize seed (Lamphear et al., 2005), tomato (Drake, WO9925835), corn (Nikolov, WO0121270), fruits and vegetables (WO9742333), mice (Yan et al., 2013).

In spite of the above disclosures, there is still a need for new methods and biological systems for efficient and low-cost production of sweet proteins as a source of alternative sweeteners that have low or no calories. It is also desirable for low cost and popular plants that produce low- or non-calorie sweet proteins as sources for healthy flavor, sweetener, consumable, food, or beverage products. It is against the above background that the present disclosure presents advantages and advancements to address this need.

### SUMMARY OF DISCLOSURE

The present disclosure includes a solution to producing a sweet protein or a variant thereof having low or no calorie. By using genome editing, and/or recombinant DNA, and/or plant transformation techniques, non-native genes encoding a sweet protein are generated or introduced or implemented in the genome of a plant, thereby forming a genetically modified plant, wherein the plant by the native genome thereof prior to modification may not produce the sweet protein naturally. Such genetically modified plant and a progeny thereof are enabled to produce non-native sweet protein and/or a variant(s) thereof.

The provided solution is of significant advantage. First, the production of sweet proteins in plants may have better techno-economics because of mature agricultural technologies. In addition, the solution may allow for sweet protein production throughout more parts of the plant and not just within fruits, thereby enhancing the entire nutritional and economic value of the plants. Moreover, generating or implementing genes responsible for producing sweet protein into fast-growing or fast-maturing plants/crops may improve efficiencies of production and processing of sweet proteins, and provide cost-effective benefits. The provided solution may allow for production of various foodstuff derived from the plant described herein. Such foodstuff includes but is not limited to sweeteners, full purity sweetener, sweetening compositions, juices, low purity juices, higher purity extracts, solid or semi-solid foods, beverages, or consumables. The foodstuff according to the present disclosure may advantageously provide novel flavor, taste improvement, unique palatability profile, and/or low- or non-calorie by using these sweet protein producing plants and materials or parts thereof.

It should be noted that previous disclosures primarily focused on genetically engineered microorganism (mostly bacteria or yeast) based method for making sweet proteins. The present disclosure distinctly describes a genetically modified or transgenic or gene-edited plant that is enabled to produce non-native sweet proteins. More importantly, sweeteners and food products derived from plants could also be more acceptable to consumers than synthetic or microorganism-generated sweeteners. The provided plants herein may allow for the production of flavor or sweetener or food or juice or plant extracts or plant materials or other derived consumables with a lower ratio of calories to sweetness that could be used with less processing or with preferred additional flavor characteristics.

It is important to note that the ability of a transgenic or gene-edited plant comprising a genomic transformation event to produce fruits and/or seeds is rare. Surprisingly, the plants according to the present disclosure produced various tissues including fruits and seeds, wherein the various tissues including fruits and seeds all comprise and produce non-native sweet proteins. The sweet protein containing fruit of the present plants can be used as a source for various food and beverage products and therefore provides techno-economic advantages in food and consumable industry. In addition, the seed-producing plants of the present disclosure can benefit mass production as well as cost-effective production of sweet proteins by propagation of the seeds and agricultural reproduction of the plants using various plant-breeding technologies.

Watermelon fruit has great potential for production of low- and/or non-caloric sweeteners due to its large size and popular flavor. Watermelon by its natural genome does not produce known native sweet protein. The present disclosure advantageously provides an effective approach for tissue-specific expression of non-native sweet proteins in various parts of watermelon by employing genomic modification strategies. Sweet proteins that are specifically expressed in the edible portion of watermelon fruit can be generated.

The present disclosure generally relates to a plant comprising a genomic transformation event, wherein the genomic transformation event enables the plant to produce a non-native expression or concentration of a sweet protein. In some embodiments, the plant is a transgenic plant, and the genomic transformation event is obtained by plant transformation techniques. In other embodiments, the plant is a gene edited plant, and the genomic transformation event is obtained by gene or genome editing techniques.

In some embodiments, the genomic transformation event comprises one or more of the nucleotide sequences encoding the sweet protein. The one or more of the nucleotide sequences may be implemented within the genome of the plant by an expression cassette, wherein the expression cassette comprises the nucleotide sequences encoding the sweet protein. In certain embodiments, the genomic transformation event is added to the plant by transforming the plant with the sweet protein producing nucleotide sequences.

In some embodiments, the genomic transformation event or the expression cassette comprises one or more of the nucleotide sequences as set forth in SEQ ID NOs. 1-24. In certain embodiments, the nucleotide sequences encoding the sweet protein have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-24.

In some embodiments, the genomic transformation event or the expression cassette comprises one or more regulatory sequences selected from the group consisting of: promoter, spacer, epitope tag, terminator, coding sequence, signal peptide, or combinations thereof. In certain embodiments, the regulatory sequences have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-6 and 8-13.

In some embodiments, the genomic transformation event or the expression cassette comprises a promotor operably linked with the nucleotide sequence(s) encoding the sweet protein.

In some embodiments, the genomic transformation event or the expression cassette comprises a start codon operably linked with the nucleotide sequence(s) encoding the sweet protein.

In some embodiments, the genomic transformation event or the expression cassette comprises a nucleotide sequence encoding a signal peptide, wherein the nucleotide sequence encoding a signal peptide is operably linked with the nucleotide sequence(s) encoding the sweet protein.

In some embodiments, the genomic transformation event or the expression cassette further comprises a reporter gene.

The sweet proteins according to the present disclosure include but are not limited to thaumatin, monellin, mabinlin, brazzein, egg white lysozyme, neoculin, or variant thereof, or combinations thereof. In some embodiments, the sweet protein is brazzein or a variant thereof. As an exemplary example, the brazzein according to the present disclosure is des-pyrE-bra. The amino acid sequence of des-pyrE-bra is set forth in SEQ ID NO: 25.

In some embodiments, the sweet protein according to the present disclosure comprises an amino acid sequence having at least 70%, at least 75%, at least 80%, at least 85%, at least 90%, at least 95%, at least 96%, at least 97%, at least 98%, or at least 99% to the amino acid sequence set forth in SEQ ID NO: 25.

In some embodiments, the present disclosure relates to a plant part obtainable from the described plant comprising a genomic transformation event, wherein the genomic transformation event enables the plant to produce a non-native expression of concentration of a sweet protein.

In some embodiments, a progeny or an ancestor of the described plant is a source of the genomic transformation event enabling the progeny and the ancestor to produce the sweet protein.

In some embodiments, the plant is a member of Cucurbitacene or Curcubits plant family. In a particular embodiment, the plant is a watermelon.

In some embodiments, the present disclosure relates to foodstuff comprising the sweet protein produced by the plant described herein. The foodstuff may be a sweetener, flavor, food, beverage, or food ingredient.

In some aspects, the present disclosure relates to a method of making a genetically modified plant described herein. The method comprises combining a plant with a genomic transformation event, wherein the genomic transformation event enables the genetically modified plant to produce a non-native expression or concentration of the sweet protein. In some embodiments, the method further comprises: preparing/providing plasmids comprising an expression cassette, wherein the expression cassette expresses the non-native sweet protein; transforming a host cell with the plasmids; and transfecting the plant with a plurality of the transformed host cell, wherein the genetically modified plant is a transgenic plant. In other embodiments, the genomic transformation event is obtained by a method of genome editing, and wherein the genetically modified plant is a gene-edited plant.

In some aspects, the present disclosure relates to a biosynthetic method for producing a non-native sweet protein described herein. The biosynthetic method includes: (a) combining a plant with a genomic transformation event forming a genetically modified plant, wherein the genomic transformation event enables the genetically modified plant to produce a non-native expression or concentration of the sweet protein; (b) growing and regenerating a population of the genetically modified plant: (c) selecting the genetically modified plants that produce the sweet protein; and (d) harvesting the sweet protein. In some embodiments, the method further comprises: preparing/providing plasmids comprising an expression cassette, wherein the expression cassette expresses the non-native sweet protein; transforming a host cell with the plasmids; and transfecting the plant with a plurality of the transformed host cell, wherein the genetically modified plant is a transgenic plant.

### Definition and Interpretation of Selected Terms

The following definitions or interpretations of technical terms will be used throughout the present disclosure. The technical terms used herein are generally to be given the meaning commonly applied to them in the pertinent art of plant biology, molecular biology, bioinformatics, and plant breeding. All of the following term definitions apply to the complete content of this application. It is to be understood that as used in the specification and in the claims, “a” or “an” can mean one or more, depending upon the context in which it is used. Thus, for example, reference to “a cell” can mean that at least one cell can be utilized.

The term “essentially,” “about,” “approximately” and the like in connection with an attribute or a value, particularly also define exactly the attribute or exactly the value, respectively. The term “about” in the context of a given numeric value or range relates in particular to a value or range that is within 20%, within 10%, or within 5% of the value or range given. As used herein, the term “comprising” also encompasses the term “consisting of.”

The terms “peptides,” “oligopeptides,” “polypeptide,” “protein”, or “enzyme” are used interchangeably herein and refer to amino acids in a polymeric form of any length, linked together by peptide bonds, unless mentioned herein otherwise.

The terms “gene sequence(s).” “polynucleotide(s),” “nucleic acid sequence(s),” “nucleotide sequence(s),” “nucleic acid(s),” “nucleic acid molecule” are used interchangeably herein and refer to nucleotides, either ribonucleotides or deoxyribonucleotides or a combination of both, in a polymeric unbranched form of any length.

### Transgenic/Transgene/Recombinant Gene

For the purposes of the present disclosure, “transgenic,” “transgene,” or “recombinant” means with regard to, for example, a nucleic acid sequence, an expression cassette, genetic construct, or a vector comprising the nucleic acid sequence or an organism transformed with the nucleic acid sequences, expression cassettes or vectors according to the disclosure, all those constructions brought about by recombinant methods in which either (a) the sequences of the nucleic acids or a part thereof, or (b) genetic control sequence(s) which is operably linked with the nucleic acid sequence according to the disclosure, for example a promoter, or (c) combinations of (a) and (b), are not located in their natural genetic environment or have been modified by recombinant methods e.g. modified and/or inserted artificially by genetic engineering methods.

As used herein, the term “transgenic” relates to an organism e.g. transgenic plant refers to an organism, e.g., a plant, plant cell, callus, plant tissue, or plant part that exogenously contains the nucleic acid, construct, vector, or expression cassette described herein or a part thereof which is partially or fully integrated into the propagatable genome of the plant by recombinant processes such as Agrobacteria-mediated transformation or particle bombardment.

### Plant/Genetically Modified Plant/Transgenic Plant/Gene Edited Plant/Genome Edited Plant/Natural Plant

The term “plant” as used herein encompasses whole plants, ancestors and progeny of the plants and plant parts, including seeds, shoots, stems, leaves, roots (including tubers), flowers, and tissues and organs, wherein each of the aforementioned comprise the gene/nucleic acid of interest. The term “plant” also encompasses plant cells, suspension cultures, callus tissue, embryos, meristematic regions, gametophytes, sporophytes, pollen and microspores, again wherein each of the aforementioned comprises the gene/nucleic acid of interest.

The term “genetically modified plant” refers to a plant comprising at least one cell genetically modified by man A genetically modified plant and a corresponding unmodified plant as used herein refer to a plant comprising at least one genetically modified cell and to a plant of the same type lacking said modification, respectively.

One of ordinary skill in the art would appreciate that a genetically modified plant may encompass a plant comprising at least one cell genetically modified by man. In some embodiments, the genetic modification includes modification of an endogenous gene(s), for example by introducing mutation(s) deletions, insertions, transposable element(s) and the like into an endogenous polynucleotide or gene of interest. Additionally, or alternatively, in some embodiments, the genetic modification includes transforming at least one plant cell with a heterologous polynucleotide or multiple heterologous polynucleotides. The skilled artisan would appreciate that a genetically modified plant comprising transforming at least one plant cell with a heterologous polynucleotide or multiple heterologous polynucleotides may in certain embodiments be termed a transgenic plant.

A comparison of a genetically modified plant to a corresponding unmodified plant as used herein encompasses comparing a plant comprising at least one genetically modified cell and to a plant of the same type lacking the modification. One of ordinary skill in the art would appreciate that the term transgenic when used in reference to a plant as disclosed herein, encompasses a plant that contains at least one heterologous polynucleotide transcribed in one or more of its cells. The term transgenic material encompasses broadly a plant or a part thereof, including at least one cell, multiple cells or tissues that contain at least one heterologous polynucleotide in at least one of cell. Thus, comparison of a “transgenic plant” and a “corresponding non transgenic plant”, or of a “genetically modified plant comprising at least one cell having altered expression, wherein said plant comprising at least one cell comprising a heterologous transcribable polynucleotide” and a “corresponding unmodified plant” encompasses comparison of the “transgenic plant” or “genetically modified plant” to a plant of the same type lacking said heterologous transcribable polynucleotide. A skilled artisan would appreciate that, in some embodiments, a “transcribable polynucleotide” comprises a polynucleotide that can be transcribed into an RNA molecule by an RNA polymerase.

### Endogenous/Native

An “endogenous” or “native” nucleic acid and/or a protein refers to the a nucleic acid and/or a protein in question as found in a plant in its natural form (i.e., without there being any human intervention like recombinant DNA engineering technology), but also refers to that same gene (or a substantially homologous nucleic acid/gene) in an isolated form subsequently (re)introduced into a plant (a transgene). A transgenic plant containing such a transgene may or may not encounter a substantial reduction of the transgene expression and/or substantial reduction of expression of the endogenous gene.

### Exogenous

The term “exogenous” (in contrast to “endogenous”) nucleic acid or gene refers to a nucleic acid that has been introduced in a plant by means of recombinant DNA technology. An “exogenous” nucleic acid can either not occur in a plant in its natural form, be different from the nucleic acid in question as found in a plant in its natural form, or can be identical to a nucleic acid found in a plant in its natural form, but integrated not within its natural genetic environment. The corresponding meaning of “exogenous” is applied in the context of protein expression. For example, a transgenic plant containing a transgene, i.e., an exogenous nucleic acid, may, when compared to the expression of the endogenous gene, encounter a substantial increase of the expression of the respective gene or protein in total. A transgenic plant according to the present disclosure includes one or more exogenous nucleic acids integrated at any genetic loci and optionally the plant may also include the endogenous gene within the natural genetic background.

### Expression Cassette

“Expression cassette” as used herein is a vector DNA capable of being expressed in a host cell. The DNA, part of the DNA or the arrangement of the genetic elements forming the expression cassette can be artificial. The skilled artisan is aware of the genetic elements that must be present in the expression cassette in order to be successfully yield expression. The expression cassette comprises a sequence of interest to be expressed operably linked to one or more control sequences (at least to a promoter) as described herein. Additional regulatory elements may include transcriptional as well as translational enhancers. Those skilled in the art will be aware of terminator and enhancer sequences that may be suitable for use in performing the disclosure. An intron sequence may also be added to the S′ untranslated region (UTR) or in the coding sequence to increase the amount of the mature message that accumulates in the cytosol, as described in the definitions section for increased expression/overexpression. Other control sequences (besides promoter, enhancer, silencer, intron sequences, 3′UTR and/or S′UTR regions) may be protein and/or RNA stabilizing elements. Such sequences would be known or may readily be obtained by a person skilled in the art.

The expression cassette may be integrated into the genome of a host cell and replicated together with the genome of said host cell.

### Vector

Vector or vector construct is DNA (such as but, not limited to plasmids, viral DNA, and chromosome vector) artificial in part or total or artificial in the arrangement of the genetic elements contained-capable of replication in a host cell and used for introduction of a DNA sequence of interest into a host cell or bost organism. A vector may be a construct or may comprise at least one construct. A vector may replicate without integrating into the genome of a host cell, e.g. a plasmid vector in a bacterial host cell, or it may integrate part or all of its DNA into the genome of the host cell and thus lead to replication and expression of its DNA Host cells of the disclosure may be any cell selected from bacterial cells, such as Escherichia coli or Agrobacterium species cells, yeast cells, fungal, algal or cyanobacterial cells, or plant cells. The skilled artisan is aware of the genetic elements that must be present on the genetic construct in order to successfully transform, select and propagate host cells containing the sequence of interest. Typically, the vector comprises at least one expression cassette. The one or more sequence(s) of interest is operably linked to one or more control sequences (at least to a promoter) as described herein. Additional regulatory elements may include transcriptional as well as translational enhancers. Those skilled in the art will be aware of terminator and enhancer sequences that may be suitable for use in performing the herein disclosed techniques.

### Operably Linked

The term “operably linked” or “functionally linked” is used interchangeably and, as used herein, refers to a functional linkage between the promoter sequence and the gene of interest, such that the promoter sequence is able to direct transcription of the gene of interest.

### Regulatory/Control Sequence

The term “regulatory or control sequence” is defined herein to include all components necessary for the expression from a polynucleotide encoding a sweet protein of the present disclosure. Each control sequence may be native or foreign to the nucleotide sequence encoding the sweet protein in nature or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleotide sequence encoding a sweet protein

### Coding Sequence

When used herein the term “coding sequence” means a polynucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, RNA, synthetic, or recombinant nucleotide sequence.

### Promoter/Plant Promoter/Strong Promoter/Weak Promoter

A “promoter” or “plant promoter” comprises regulatory elements, which mediate the expression of a coding sequence segment in plant cells. The “plant promoter” can originate from a plant cell, e.g. from the plant which is transformed with the nucleic acid sequence to be expressed in the present systems and described herein. This also applies to other “plant” regulatory signals, such as plant terminators. The promoters upstream of the nucleotide sequences useful in the methods of the present disclosure can be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoters is increased by modification of their sequence, or that they are replaced completely by more active promoters, even promoters from heterologous organisms. For expression in plants, the nucleic acid molecule must, as described herein, be linked operably to or comprise a suitable promoter which expresses the gene at the right point in time and with the required spatial expression pattern.

The promoter used herein broadly encompasses constitutive promoter, ubiquitous promoter, developmentally-regulated promoter, inducible promoter, organ-specific promoter, tissue-specific promoter, seed-specific promoter, green-tissue specific promoter, meristem-specific promoter, etc. A “ubiquitous promoter” is active in substantially all tissues or cells of an organism.

For the identification of functionally equivalent promoters, the promoter strength and/or expression pattern of a candidate promoter may be analyzed for example by operably linking the promoter to a reporter gene and assaying the expression level and pattern of the reporter gene in various tissues of the plant. Generally, by “weak promoter” is intended a promoter that drives expression of a coding sequence at a low level. By “low level” is intended at levels of about 1/10,000 transcripts to about 1/100,000 transcripts, to about 1/500,0000 transcripts per cell. Conversely, a “strong promoter” drives expression of a coding sequence at high level, or at about 1/10 transcripts to about 1/100 transcripts to about 1/1000 transcripts per cell. Generally, by “medium strength promoter” is intended a promoter that drives expression of a coding sequence at a lower level than a strong promoter.

### Terminator

The term “terminator” encompasses a control sequence which is a DNA sequence at the end of a transcriptional unit which signals 3′ processing and polyadenylation of a primary transcript and termination of transcription. The terminator can be derived from the natural gene, from a variety of other plant genes, or from T-DNA. The terminator to be added may be derived from, for example, the nopaline synthase or octopine synthase genes, or alternatively from another plant gene, or less preferably from any other eukaryotic gene.

### Reporter Gene

“Selectable marker,” “selectable marker gene,” or “reporter gene” includes any gene that confers a phenotype on a cell in which it is expressed to facilitate the identification and/or selection of cells that are transfected or transformed with a nucleic acid construct of the disclosure. These marker genes enable the identification of a successful transfer of the nucleic acid molecules via a series of different principles. Suitable markers may be selected from markers that confer antibiotic or herbicide resistance, that introduce a new metabolic trait or that allow visual selection. Examples of selectable marker genes include genes conferring resistance to antibiotics such as kanamycin (KAN) or hygromycin (Hyg). Expression of visual marker genes results in the formation of fluorescence (Green Fluorescent Protein, GFP; Red Fluorescent Protein, RFP; and derivatives thereof). This list represents only a small number of possible markers. A skilled artisan is familiar with such markers. Different markers are preferred, depending on the organism and the selection method.

### Expression/Gene Expression

The term “expression” or “gene expression” means the transcription of a specific gene or specific genes or specific genetic construct. The term “expression” or “gene expression” in particular means the translation of the RNA and therewith the synthesis of the encoded protein/enzyme, i.e., protein/enzyme expression.

### Percent Identity/Homology

As used herein, sequence identity, homology, or “percent identity” means the extent to which two optimally aligned DNA or protein segments are invariant throughout a window of alignment of components, for example nucleotide sequence or amino acid sequence. An “identity fraction” for aligned segments of a test sequence and a reference sequence is the number of identical components that are shared by sequences of the two aligned segments divided by the total number of sequence components in the reference segment over a window of alignment which is the smaller of the full test sequence or the full reference sequence. “Percent identity” (“% identity”) is the identity fraction times 100.

### Introduction/Implementation/Transformation

The term “introduction,” “implementation,” or “transformation” as referred to herein encompasses the transfer of an exogenous polynucleotide into a host cell, irrespective of the method used for transfer.

Plant tissue capable of subsequent clonal propagation, whether by organogenesis or embryogenesis, may be transformed with a genetic construct of the present disclosure and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The polynucleotide may be transiently or stably introduced into a host cell and may be maintained non-integrated, for example, as a plasmid. Alternatively, it may be integrated into the host genome. The resulting transformed plant cell may then be used to regenerate a transformed plant in a manner known to persons skilled in the art. Alternatively, a plant cell that cannot be regenerated into a plant may be chosen as host cell, i.e. the resulting transformed plant cell does not have the capacity to regenerate into a (whole) plant.

The transfer of foreign genes into the genome of a plant is called transformation. Transformation of plant species is now a fairly routine technique. Advantageously, any of several transformation methods may be used to introduce the gene of interest into a suitable ancestor cell. The methods described for the transformation and regeneration of plants from plant tissues or plant cells may be utilized for transient or for stable transformation. Transformation methods include the use of liposomes, electroporation, chemicals that increase free DNA uptake, injection of the DNA directly into the plant, particle gun bombardment, transformation using viruses or pollen and microprojection. Transgenic plants, including transgenic crop plants, are preferably produced via Agrobacterium-mediated transformation. An advantageous transformation method is the transformation in planta. To this end, it is possible, for example, to allow the agrobacteria to act on plant seeds or to inoculate the plant meristem with agrobacteria. It has proved particularly expedient in accordance with the disclosure to allow a suspension of transformed agrobacteria to act on the intact plant or at least on the flower primordia. The plant is subsequently grown on until the seeds of the treated plant are obtained (Clough and Bent, Plant J. (1998) 16, 735-743). The nucleic acids or the construct to be expressed is preferably cloned into a vector, which is suitable for transforming Agrobacterium tumefaciens, for example pBin19 (Bevan et al., Nucl. Acids Res. 12 (1984) 8711). Agrobacteria transformed by such a vector can then be used in known manner for the transformation of plants, sach as plants used as a model, like Arabidopsis (Arabidopsis thaliana is within the scope of the present disclosure not considered as a crop plant), or crop plants such as, by way of example, tobacco plants, for example by immersing bruised leaves or chopped leaves in an agrobacterial solution and then culturing them in suitable media. The transformation of plants by means of Agrobacterium tumefaciens is described, for example, by Hofgen and Willmitzer in Nucl. Acid Res. (1988) 16, 9877 or is known inter alia from F. F. White, Vectors for Gene Transfer in Higher Plants; in Transgenic Plants, Vol. 1, Engineering and Utilization, eds. S. D. Kung and R. Wu, Academic Press, 1993, pp. 15-38.

### Ploidy/Ploidy Level/Chromosomal Ploidy/Polyploidy

Ploidy or chromosomal ploidy refers the number of complete sets of chromosomes occurring in the nucleus of a cell. Somatic cells, tissues, and individual organisms can be described according to the number of sets of chromosomes present (the “ploidy level”): monoploid (1 set), diploid (2 sets), triploid (3 sets), tetraploid (4 sets), pentaploid (5 sets), hexaploid (6 sets), heptaploid or septaploid (7 sets), etc. The generic term polyploidy is used herein to describe cells with three or more chromosome sets.

### Modulation

The term “modulation” means in relation to expression or gene expression, a process in which the expression level is changed by said gene expression in comparison to the control plant, the expression level may be increased or decreased. The original, unmodulated expression may be of any kind of expression of a structural RNA (IRNA, tRNA) or mRNA with subsequent translation. For the purposes of this application, the original unmodulated expression may also be absence of any expression. The term “modulating the activity” or the term “modulating expression” shall mean any change of the expression of the target nucleic acid sequences and/or encoded proteins, which leads to increased or decreased yield-related trait(s) such as but not limited to increased or decreased seed yield and/or Increased or decreased growth of the plants. The expression can increase from zero (absence of, or immeasurable expression) to a certain amount, or can decrease from a certain amount to immeasurable small amounts or zero.

Generally, after transformation, plant cells or cell groupings are selected for the presence of one or more markers which are encoded by plant-expressible genes co-transferred with the gene of interest, following which the transformed material is regenerated into a whole plant. To select transformed plants, the plant material obtained in the transformation is, as a rule, subjected to selective conditions so that transformed plants can be distinguished from untransformed plants. For example, the seeds obtained in the above described manner can be planted and, after an initial growing period, subjected to a suitable selection by spraying. A further possibility consists in growing the seeds, if appropriate after sterilization, on agar plates using a suitable selection agent so that only the transformed seeds can grow into plants. Alternatively, the transformed plants are screened for the presence of a selectable marker such as the ones described herein.

Following DNA transfer and regeneration, putatively transformed plants may also be evaluated, for the presence of the gene of interest, copy number and/or genomic organization.

The generated transformed plants may be propagated by a variety of means, such as by clonal propagation or classical breeding techniques. For example, a first generation (or TH) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette): grafts of transformed and untransformed tissues (e.g., in plants, a transformed rootstock grafted to an untransformed scion).

As described herein a plant, plant part, seed or plant cell transformed with, or interchangeably transformed by, a construct or transformed with or by a nucleic acid is to be understood as meaning a plant, plant part, seed or plant cell that carries said construct or said nucleic acid as a transgene due the result of an introduction of said construct or said nucleic acid by biotechnological means. The plant, plant part, seed or plant cell therefore comprises said expression cassette, said recombinant construct, or said recombinant nucleic acid.

### BRIEF DESCRIPTION OF THE DRAWINGS

FIG. 1 shows some examples of brazzein mutation and their reported effect on sweetness. NS=completely non-sweet; RS=reduced sweetness: NC=no change; IS=increased sweetness; Max=maximally increased sweetness. Color legend: pink=deletion of the N- or C-terminus; orange=larger residue change to alanine; light blue=change of side-chain size; yellow=change of charge; green=removal or shift of disulfide bond. In red are the amino acid residues identified as most important for the sweetness elicitation.

FIG. 2 shows the detection of brazzein-FLAG protein in the protoplast culture media according to Example 1. Anti-FLAG antibody detected a peak at around 6-7 kDa, from the culture media of protoplast transfected with the expression cassette design #4 (BAAS Des-pyrE-bra FLAG) of Table 1, but not from mock (empty) or GFP controls. The protein could also be detected at much smaller quantity (about 15%) from the protoplasts transfected with the same expression cassette. This is a representative graph from three independent replicates.

### DETAILED DESCRIPTION

### Construction of Genomic Transformation Event

The present disclosure generally relates to a plant comprising a genomic transformation event, wherein the genomic transformation event enables the plant to produce a non-native expression or concentration of a sweet protein. In some embodiments, the plant is a transgenic plant or a genetically modified plant, and the genomic transformation event comprises an expression cassette, wherein the expression cassette comprises one or more of the nucleotide sequences encoding the sweet protein. In other embodiments, the plant is a gene-edited plant, and the genomic transformation event is obtained by gene or genome editing techniques.

In some embodiments, the genomic transformation event comprises one or more nucleotide sequences encoding the sweet protein. The one or more of the nucleotide sequences may be implemented into the genome of the plant by an expression cassette, wherein the expression cassette comprises the nucleotide sequences encoding the sweet protein. In certain embodiments, the genomic transformation event is added to the plant by transforming the plant with the sweet protein producing nucleotide sequences.

In some embodiments, the sweet protein is thaumatin or a variant thereof, monellin or a variant thereof, mabinlin or a variant thereof, brazzein or a variant thereof, egg white lysozyme or a variant thereof, pentadin or a variant thereof, neoculin or a variant thereof, or any combinations thereof.

In some embodiments, the sweet protein consists of brazzein or a variant thereof. Brazzein according to the present disclosure encompasses the wild type and all forms and folding configurations thereof. Brazzein can be found in different forms in nature. The minor form, called des-pyrE-bra, which lacks the N-terminal pyroglutamic acid (pyrE) residue is sweeter than the major form (with pyrE). As an exemplary example, the brazzein according to the present disclosure is des-pyrE-bra. The amino acid sequence of des-pyrE-bra is set forth in SEQ ID NO: 25.

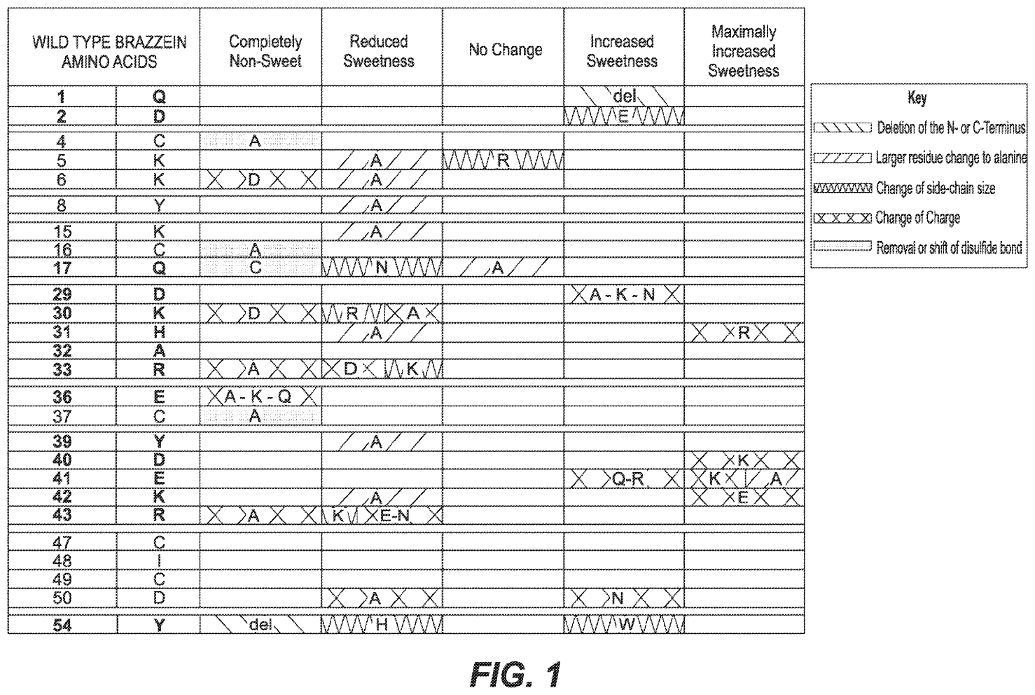

Brazzein according to the present disclosure encompasses all mutants thereof. A mutant may comprise mutations, deletions, alterations, or additions of atom(s) or functional groups or residues or electrical charges or radicals of one or more positions of the amino acid sequence of the wild type brazzein. Examples of brazzein mutants include but are not limited to mutations in D29A, D29K, D29N, E41K, A2ins, D2N. Q17A, K6, K30, R33, E36, R43, the deletion of the C-term YS4 amino acid, mutants in the KS, Y8, K15, H31, and D50 residues, mutations of the negatively charged D29 to neutral or positively charged residues, mutations of residues 29-33, 39-43, and 36, positive charge in the 29-33 region. Other exemplary examples of brazzein mutants are shown in FIG. 1. Accordingly, in some embodiments, the sweet protein according to the present disclosure comprises an amino acid sequence having at least 70%, at least 75%, at least 80%, at least 85%, at least 90%, at least 91%, at least 92%, at least 30%, at least 94%, at least 95%, at least 96%, at least 97%, at least 98%, or at least 99% to the amino acid sequence set forth in SEQ ID NO: 25.

In some embodiments, the genomic transformation event comprises an expression cassette, wherein the expression cassette comprises one or more of the nucleotide sequences as set forth in SEQ ID NOs: 1˜24. In certain embodiments, the nucleotide sequences have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-24.

In some embodiments, the genomic transformation event comprises one or more of the nucleotide sequences encoding a sweet protein. The sweet protein described herein include thaumatin, monellin, mabinlin, brazzein, egg white lysozyme, neoculin, or combinations thereof. The “nucleotide sequences encoding a sweet protein” used herein encompass nucleotide sequences encoding a polypeptide that have one or more amino acid sequences of a sweet protein. As an exemplary example, the nucleotide sequence set forth in SEQ ID NO: 7 is capable of encoding brazzein. The nucleotide sequences set forth in SEQ ID NOs: 14-24 are capable of encoding a polypeptide that have one or more amino acid sequences of brazzein. In some embodiments, the nucleotide sequences set forth in SEQ ID NOs: 7 and 14-24 are capable of encoding a polypeptide that has at least 70%, at least 75%, at least 80%, at least 85%, at least 90%, at least 91%, at least 92%, at least 30%, at least 94%, at least 95%, at least 96%, at least 97%, at least 98%, or at least 99% to the amino acid sequence set forth in SEQ ID NO 25.

In some embodiments, the genomic transformation event or the expression cassette comprises one or more regulatory sequences selected from the group consisting of: promoter, spacer, epitope tag, terminator, coding sequence, signal peptide, or combinations thereof. In certain embodiments, the regulatory sequences have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-6 and 8-13. The regulatory sequences may be operably linked with the nucleotide sequence(s) encoding the sweet protein.

In certain embodiments, the genomic transformation event or the expression cassette comprises one or more nucleotide sequences encoding an epitope tag, wherein the one or more nucleotide sequences have one or more nucleotide sequences set forth in SEQ ID NO: 8. In other embodiments, the one or more epitope tags has one or more nucleotide sequences having a sequence identity of at least 70%, at least 75%, at least 80%, at least 85%, at least 90%, at least 95%, at least 96%, at least 97%, at least 98%, or at least 99% to the nucleotide sequences set forth in SEQ ID NO: 8. An exemplary example of the epitope tag is FLAG having an amino acid sequence set forth in SEQ ID NO: 31. Accordingly, in some embodiments, the present plant is enabled to produce a non-native polypeptide comprising an amino acid sequence of a sweet protein operably linked to an amino acid sequence of an epitope tag as set forth in SEQ ID NO: 31.

In some embodiments, the sweet protein is encoded with a propeptide in the N-terminus. A protein including a propeptide is generally immature and probably non-functional and can be converted to a mature functional protein by catalytic or autocatalytic cleaving off of the propeptide. In certain embodiments, the genomic transformation event or the expression cassette comprises the nucleotide sequences encoding the sweet protein operably linked to a nucleotide sequence encoding a propeptide.

In some embodiments, the sweet protein is encoded with a signal peptide in the N-terminus. Where both signal peptide and propeptide sequences are present at the N-terminus of a protein, the propeptide sequence is positioned next to the N-terminus of the mature protein and the signal peptide sequence is positioned next to the N-terminus of the propeptide sequence. The signal peptide is cleaved off by the host cell of the plant. Preferably, it is cleaved off by the host cell before, during or immediately after secretion. In certain embodiments, the genomic transformation event or the expression cassette further comprises one or more nucleotide sequences encoding a signal peptide operably linked to the nucleotide sequences encoding the sweet protein, wherein the nucleotide sequences have one or more sequences set forth in SEQ ID NO: 9-13. In other embodiments, the nucleotide sequences encoding the signal peptide has one or more nucleotide sequences having a sequence identity of at least 70%, at least 75%, at least 80%, at least 85%, at least 90%, at least 95%, at least 96%, at least 97%, at least 98%, or at least 99% to the nucleotide sequences set forth in SEQ ID NO: 9-13. In certain embodiments, the nucleotide sequences encoding a signal peptide or a propeptide or both that are operably linked with the nucleotide sequences encoding the sweet protein.

As an exemplary example, addition of a N-terminal secretion signal peptide to the amino acid sequence of brazzein could mediate translocation of brazzein across the cell membrane, which results in cleavage of the secretion signal leading to apoplastic accumulation of brazzein. Examples of signal peptides described herein include BAAS, PR1a, CHIA, BP80, and S2S. The amino acid sequence of each of BAAS, PR1a, CHIA, BP80, and S2S are set forth in SEQ ID NO: 26-30, respectively. Accordingly, in some embodiments, the present plant produces a non-native polypeptide comprising an amino acid sequence of a sweet protein operably linked to an amino acid sequence of a signal peptide as set forth in SEQ ID NO: 26-30.

In some embodiments, the genomic transformation event or the expression cassette comprises one or more coding sequences. As an exemplary example, the amino acid sequence of the sweet protein, such as des-pyrE-bra, does not start with a methionine residue. A novel start codon ATG is added to the sequence to produce a protein that varies from the original by a single amino acid. The sequence involving a start codon is expected to serve as a valuable scientific reagent for rapid testing and optimization of expression systems. Exemplary examples of the codon optimized nucleotide sequences encoding a sweet protein are set forth in SEQ ID NOs: 14-24. In some embodiments, the genomic transformation event or the expression cassette comprises one or more nucleotide sequences having a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 14-24.

Table 1 shows some non-limiting example designs of the expression cassettes according to the present disclosure. Each of the genes of interest, for example, the nucleotide sequence encoding brazzein, is operably linked to a promoter sequence, and/or a nucleotide sequence encoding an epitope tag, and/or a gene sequence encoding a signal peptide, and/or a codon, thereby forming an expressible gene. Such “expressible genes” can be further modified by operably linked through space sequences (spacers) to alter expression or the enzyme product produced by the “expression cassettes.” In some embodiments, the expression cassette of the present plant comprises one or more expressible genes and one or more spacers, wherein, each expressible gene comprises one or more nucleotide sequences encoding a sweet protein.

TABLE 1
Design Scheme of Brazzein Expression Cassettes.

| | | Signal | Epitope | Codon | |
|---|---|---|---|---|---|
| Sample | Expression cassette | Peptide | Tag | Usage | Promoter |
| 1 | des-pyrE-Brazzein | N/A | N/A | WM | CsVMV |
| 2 | BAAS-des-pyrE-Brazzein | BAAS | N/A | WM | CsVMV |
| 3 | PR1a-des-pyrE-Brazzein | PR1 | N/A | WM | CsVMV |
| 4 | BAAS-des-pyrE-Brazzein- | BAAS | FLAG | WM | CsVMV |
| | FLAG | | | | |
| 5 | PR1a-des-pyrE-Brazzein- | PR1 | FLAG | WM | CsVMV |
| | FLAG | | | | |
| 6 | CHIA-des-pyrE-Brazzein- | CHIA | FLAG | WM | CsVMV |
| | FLAG | | | | |
| 7 | BP80-des-pyrE-Brazzein- | BP80 | FLAG | WM. | CsVMV |
| | FLAG | | | | |
| 8 | S2S-des-pyrE-Brazzein- | S2S | FLAG | WM | CsVMV |
| | FLAG | | | | |
| 9 | des-pyrE-Brazzein-FLAG | N/A | FLAG | WM | CsVMV |
| 10 | des-pyrE-Brazzein-FLAG | N/A | FLAG | Maize | CsVMV |
| | Maize | | | | |
| 11 | des-pyrE-Brazzein-FLAG | N/A | FLAG | Canola | CsVMV |
| | Canola | | | | |
| 12 | des-pyrE-Brazzein-FLAG | N/A | FLAG | Tobacco | CsVMV |
| | Tobacco | | | | |
| 13 | des-pyrE-Brazzein-FLAG | N/A | FLAG | Tomato | CsVMV |
| | Tomato | | | | |
| 14 | des-pyrE-Brazzcin-FLAG | N/A | FLAG | Potato | CsVMV |
| | Potato | | | | |
| 15 | des-pyrE-Brazzein-FLAG | N/A | FLAG | WM | Dahlia MV |
| 16 | des-pyrE-Brazzein-FLAG | N/A | FLAG | WM | PCLSV |
| 17 | des-pyrE-Brazzein-FLAG | N/A | FLAG | WM | HLVH12 |
| 18 | des-pyrE-Brazzein-FLAG | N/A | FLAG | WM | AGPLI |

In some embodiments, the expression cassette of the present disclosure further comprises one or more reporter gene sequences encoding and expressing one or more reporter proteins. The reporter proteins include but are not limited to kanamacin resistant protein (KAN), hygromycin resistant protein (Hyg), green fluorescent protein (GFP), and green fluorescent protein (RFP).

In some embodiments, the expression cassette is carried on a plasmid so as to allow enzyme production by a host cell. In other embodiments, the expression cassette carried on a vector that allows for chromosomal integration, which allows enzymes to be expressed from a chromosome.

### Construction of Plant Lines and Transformation

In some embodiments, the method of making the plants of the present disclosure is related to constructing plant lines and combining the genomic transformation event described herein with the selected natural plant and/or transforming the selected natural plants with the expression cassettes made according to the present disclosure.

It is generally known that the native expression of brazzein is only available in Pentadiplandra brazzeana. In some embodiments of the present application, the natural plants selected to be combined or transformed with the genomic transformation event comprising the nucleotide sequences encoding brazzein are not Pentadiplandra brazzeana In particular, the natural plants prior to combination or transformation by their native genomes do not naturally produce brazzein. In certain embodiments, the selected natural plants for transformation include wild-type, or untransformed, or non-transformed Cucurbitaceae or Curcubits, which do not by its native genome naturally produce detectable brazzein. In certain embodiments, the plant is a watermelon.

In some embodiments, the plant is a fast-growing fruit or vegetable. Gene editing or transforming fast-growing economic fruits, vegetables, or plants that enable fast production of sweet proteins are of more interest with respect to efficiency and cost. Non-limiting examples of fast-growing plants are bush cherries, peaches and nectarines, apricot, radishes, plums and their relatives, sour (pic) cherries, apples, pears, sweet cherries, citrus, cucumbers, zucchinis, peas, turnips, and so on.

Genetically modified plants according to the present disclosure are produced by combining a plant with a genomic transformation event thereby forming the genetically modified plant, wherein the genomic transformation event enables the genetically modified plant to produce a non-native expression or concentration of a sweet protein described herein.

Alternatively, the genomic transformation event may be added to the plant by transforming the plant with the sweet protein producing nucleotide sequences. In some embodiments, combining the plant with the genomic transformation event is performed using one or more of the following methods: use of liposomes, use of electroporation, use of chemicals that increase free DNA uptake, use of injection of the DNA directly into the plant, use of particle gun bombardment, use of transformation using viruses or pollen, use of microprojection, or use of Agrobacterium-mediated transformation. Preferably, the transgenic plants are made via Agrobacterium-mediated transformation method. In some embodiments, the Agrobacterium Tumefaciens was transformed with the expression cassette to create a transgenic agrobacterium, which was then used to transfect the plant of interest, and the successfully transformed plants were selected based on the expression of the reporter gene in the expression cassette.

In some embodiments, the transgenic plant is transgenic watermelon (Citrullus lanatus), which was produced by the following method. Briefly, first, Agrobacterium Tumefaciens Stain EHA 105 was transformed with an expression cassette of the present application using a free-thaw method reported by Weigel et. al. (Transformation of agrobacterium using the freeze-thaw method, CSH Protoc. 2006 Dec. 1; 2006 (7)) Briefly, chemically competent agrobacterium was prepared. After addition of the expression cassette, the mixture was alternately frozen in liquid nitrogen and thawed to liquid. The cells were then allowed to recover in a Lysogeny Broth (LB) medium and plated out on LB plates with a selected antibiotic. Second, watermelon seedlings with appropriate maturity were used for preparing explants for the transformation. Cotyledons were cut off from hypocotyls, collected and appropriately treated for transformation. Then, the transformed agrobacterium culture was added to these explants. After infection, explants were blotted on sterile paper towels and transferred to plates with a Murashige and Skoog (MS) medium. The plates were sealed and allowed for co-cultivation for an appropriate period of time. After co-cultivation, the explants were moved to growth chambers to allow for growing, under the selection of the threshold content of selected antibiotics.

In other embodiments, the plant was co-transformed by infection with two or more expression cassettes, wherein the express cassettes used were selected from those shown in Table 1.

### Protein Expression in Plants and Tissues Thereof

In some embodiments, the method of making the plants of the present disclosure is related to monitoring and analyzing the expression of a sweet protein by the genomic transformation event introduced in the plants.

In some embodiments, the tissues or parts of the plants producing a non-native sweet protein made according to the present application were sampled and treated to obtain samples ready for analysis. The samples were further subject to analysis to detect the existence and/or content of the sweet protein expressed by the gene of interests in genomic transformation event or the expression cassette.

In some embodiments, the tissues of the plants producing a non-native sweet protein made according to this disclosure were grounded in a protein extraction buffer and then were subject to centrifuge. The resultant supernatant was further diluted and then were used for antibody detection. The presence of each of the target proteins were confirmed by detection of chemiluminescent signals produced by binding of corresponding antibodies, as well as the size of the proteins, as indicated by the protein size ladder used as a control in each measurement. In some embodiments, the protein detection was performed by using the Jess instrument (Bio-Techne), which automates the protein separation and immunodetection of traditional Western blotting method for protein detection. In certain embodiments, a Signal/Noise ratio (S/N ratio)>3 was used as cutoff for positive signals for the purpose of analysis and selection.

In some embodiments, sweet protein is detected in various tissues of the plants of the present application, including but not limiting to organs, tissues, leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures, placenta, locule, mesocarp, rind, epidermis, or any other part or product of the transgenic plant. In some embodiments, the plant is a watermelon, and sweet protein is detected in placenta, locule, mesocarp, rind, and epidermis thereof. In some embodiments, the expression of sweet protein is tissue-specific, for example, the expression level of the sweet protein is significantly higher in some parts or tissues of the plant comparing with other parts of tissues.

In some embodiments, the plant producing non-native sweet protein of the present disclosure is cultivatable and reproducible. A progeny or an ancestor of the transgenic plant is a source of non-native enzyme(s) enabling the progeny and the ancestor to produce the sweet protein. Propagation of the seed of the transgenic plant results in viable progeny thereof, wherein the progeny produces the non-native sweet protein or a variant thereof.

In some embodiments, the plant producing non-native sweet protein is a diploid plant, having diploid sets of chromosomes. In certain embodiments, the diploid transgenic plant produces seeds, wherein the seeds comprise the non-native sweet protein, and wherein propagation of the seeds of the diploid transgenic plant results in viable progeny thereof, wherein the progeny produces the sweet protein.

Sweeteners, Compositions, and Consumables Derived from the Plants Producing Non-Native Sweet Proteins

In some embodiments, the present disclosure relates generally to a sweetener or sweetening composition comprising a sweet protein, wherein the sweetener or sweetening composition is derived from a plant or a part thereof that produces and comprises a non-native sweet protein. In certain embodiments, the sweetener or sweetening composition is derived from the plants made according to the present disclosure. In some embodiments, the present disclosure provides a composition that comprises at least one sweetener described herein and at least one sweet protein described herein. In some embodiments, a composition comprises a sweetener component comprising at least one sweetener described herein and at least one sweet protein described herein. In one particular embodiment, a composition comprises a sweetening composition described herein, wherein the sweetening composition comprises brazzein.

The plants of the present disclosure can derive sweet protein based sweeteners upon appropriate processing. The resulting sweeteners could be used to provide low or non-caloric sweetness for many purposes. Examples of such uses to provide sweetness are in beverages, such as tea, coffee, fruit juice, and fruit beverages; foods, such as jams and jellies, peanut butter, pies, puddings, cereals, candies, ice creams, yogurts, bakery products, health care products, such as toothpastes, mouthwashes, cough drops, cough syrups; chewing gums; and sugar substitutes. In certain embodiments, the sweetener is in a juice of the plant according to the present application.

In some embodiments, the present disclosure also relates to methods of making the sweetener derived from the plants producing non-native sweet protein. The methods generally encompasses the steps including but not limited to pre-treatment cleaning and crushing of the plant or the parts thereof, extraction of the plant or the parts thereof, sedimentation and/or centrifuge, adsorption and/or separation, concentration and recovery to produce the crude sweetener, further purification, optional concentration/drying, and formulation. Means of extraction encompasses water-extraction at room temperatures, or heated temperature, or refrigerated temperature; extraction via organic solvent such as alcohol, et al. Means of separation and purification encompasses centrifuge, steeping, gravity sedimentation, filtration, micro-filtration, nano-filtration, ultra-filtration, reverse osmosis, chromatography, absorption chromatogram, high pressure liquid chromatograph (HPLC), exchanged resin purification, etc. Such techniques are generally known to those of ordinary skill in the art. A description of conventional extraction techniques for preparation of plant extracts can be found in U.S. Pat. Appl. No. 2005/0123662. In certain embodiments, the sweetener is obtained from the leaves, or fruits, or both, of the plant made according to the present disclosure.

In some embodiments, the sweetener is obtained from a watermelon producing a non-native sweet protein according to the present disclosure, wherein the sweetener comprises the non-native sweet protein produced by the watermelon. In some embodiments, the sweet protein is a brazzein.

In some embodiments, the sweet protein described above is the only sweetener in the composition or consumable, e.g. beverage. In other embodiments, a composition or consumable comprises a sweet protein described above and one or more additional sweeteners. The additional sweetener used in the sweetener component can be any known sweetener, e.g. a natural sweetener, a natural high potency sweetener, a synthetic sweetener.

Typically, the at least one sweet protein of the present disclosure comprises at least about 50% by weight of the sweetening composition, such as for example, at least about 60%, at least about 70%, at least about 80%, at least about 90% and at least about 95%. In a more particular embodiment, the at least one sweet protein of the present disclosure comprises at least about 96%, at least about 97%, at least about 98% or at least about 99% of the sweetening composition.

In some embodiments, the at least one sweet protein described herein is present in the composition in an amount such that, when the composition added to a consumable, the sweetness measured by Brix value of the consumable increases by at least 1 degree, such as, for example, at least 2 degrees Brix, at least 3 degrees Brix, at least 4 degrees Brix or at least 5 degrees Brix.

In some embodiments, the present disclosure provides a consumable comprising at least one sweetener described herein above and at least one sweet protein described herein. In some embodiments, the present disclosure provides a consumable comprising a sweetening composition comprising at least one sweetener described herein and at least one sweet protein described herein.

The at least one sweet protein described herein is typically present in the consumable in an amount effective to enhance the sweetness thereof and/or modulate one or more taste attributes of the sweetener to make the consumable taste more like a sucrose-sweetened consumable.

In some embodiments, the at least one sweet protein described herein is present in the consumable in an amount effective to provide a sweetness equivalent to about 4 degrees Brix, about 5 degrees Brix, about 6 degrees Brix, about 7 degrees Brix, about 8 degrees Brix, such as, for example, about 8 degrees Brix, about 9 degrees Brix, about degrees Brix, about 11 degrees Brix, or about 12 degrees Brix. In other embodiments, the at least one sweet protein described herein is present in the consumable in an amount effective to increase the sweetness measured by Brix value of the consumable by at least 1 degree compared to the degrees Brix of the consumable in the absence of the at least one sweet protein, such as, for example, at least 2 degrees Brix, at least 3 degrees Brix, at least 4 degrees Brix, or at least 5 degrees Brix.

In other embodiments, the at least one sweet protein described herein is present in the composition or the consumable in an amount effective such that, when the composition or the consumable is added to a consumable, one or more taste attributes of the sweetener is modulated making the consumable taste more like a sucrose-sweetened consumable compared to the same one or more taste attributes of the consumable in the absence of the at least one sweet protein. Exemplary taste attribute modulations include decreasing or eliminating bitterness, decreasing or eliminating bitter linger, decreasing or eliminating sourness, decreasing or eliminating astringency, decreasing or eliminating saltiness, decreasing or eliminating metallic notes, improving mouthfeel, decreasing or eliminating sweetness linger, and increasing sweetness onset. Multiple taste attributes of the sweetener can be modulated simultaneously, such that the consumable, overall, has more sucrose-sweetened characteristics. Methods of quantifying improvement in sucrose-sweetened characteristics are known in the art and includes, e.g., taste testing and histogram mapping.

Exemplary consumables include, but are not limited to edible gel mixes and compositions, dental compositions, foodstuffs (confections, condiments, chewing gum, cereal compositions, baked goods, dairy products, and tabletop sweetening compositions), juice (low purity juice), high purity extract, full purity sweetener, beverages, and beverage products.

In some embodiments, the present disclosure provides a beverage or beverage product derived from the plant described herein. In some embodiments, the beverage or beverage product comprises at least one sweet protein contained in or produced by the plant described herein.

“Beverage” or “Beverage product,” as used herein, is a ready-to-drink beverage, a beverage concentrate, a beverage syrup, or a powdered beverage. Suitable ready-to-drink beverages include carbonated and non-carbonated beverages. Carbonated beverages include, but are not limited to, frozen carbonated beverages, enhanced sparkling beverages, cola, fruit-flavored sparkling beverages (e.g. lemon-lime, orange, grape, strawberry and pineapple), ginger-ale, soft drinks and root beer. Non-carbonated beverages include, but are not limited to, fruit juice, fruit-flavored juice, juice drinks, nectars, vegetable juice, vegetable-flavored juice, sports drinks, energy drinks, enhanced water drinks, enhanced water with vitamins, near water drinks (e.g., water with natural or synthetic flavorings), coconut water, tea type drinks (e g. black tea, green tea, red tea, oolong tea), coffee, cocoa drink, beverage containing milk components (e.g. milk beverages, coffee containing milk components, caf au lait, milk tea, fruit milk beverages), beverages containing cereal extracts and smoothies. In one particular embodiments, the beverage or beverage product is a watermelon juice derived from a watermelon that produces non-native sweet protein according to the present disclosure.

Beverage concentrates and beverage syrups are prepared with an initial volume of liquid matrix (e.g. water) and the desired beverage ingredients. Full strength beverages are then prepared by adding further volumes of water. Powdered beverages are prepared by dry-mixing all of the beverage ingredients in the absence of a liquid matrix. Full strength beverages are then prepared by adding the full volume of water. Beverages comprise a liquid matrix, i.e. the basic ingredient in which the ingredients—including the compositions of the present disclosure—are dissolved. In one embodiment, a beverage comprises water of beverage quality as the liquid matrix, such as, for example deionized water, distilled water, reverse osmosis water, carbon-treated water, purified water, demineralized water and combinations thereof, can be used. Additional suitable liquid matrices include, but are not limited to phosphoric acid, phosphate buffer, citric acid, citrate buffer and carbon-treated water.

In some embodiments, the beverage contains an additional sweetener. The additional sweetener may or may not be derived from the plant described herein. In some embodiments, the beverage contains a carbohydrate sweetener in a concentration from about 0 to about 140,000 ppm. In some embodiments, the beverage is free or substantially from a carbohydrate sweetener that is not derived from the plant described herein.

The beverage may optionally further comprise additives including, but not limited to, carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, caffeine, flavorants and flavoring ingredients, astringent compounds, proteins or protein hydrolysates, surfactants, emulsifiers, weighing agents, juice, dairy, cereal and other plant extracts, flavonoids, alcohols, polymers and combinations thereof. Any suitable additive described herein can be used.

The beverage can further contain one or more functional ingredients, detailed above. Functional ingredients include, but are not limited to, vitamins, minerals, antioxidants, preservatives, glucosamine, polyphenols and combinations thereof. Any suitable functional ingredient described herein can be used.

In some embodiments, the present beverage is a full-calorie beverage that has up to about 120 calories per 8 oz serving. In some embodiments, the present beverage is a mid-calorie beverage that has up to about 60 calories per 8 oz serving. In some embodiments, the present beverage is a low-calorie beverage that has up to about 40 calories per 8 oz serving. In some embodiments, the present beverage is a zero-calorie that has less than about $ calories per 8 oz. serving. In some embodiments, the present beverage is a zero-calorie that has less than about 1 calorie per 8 oz. serving.

In some embodiments, the consumable according to the present disclosure is a dental composition. Dental compositions generally comprise an active dental substance and a base material. The dental composition may be in the form of any oral composition used in the oral cavity such as mouth freshening agents, gargling agents, mouth rinsing agents, toothpaste, tooth polish, dentifrices, mouth sprays, teeth-whitening agent, dental floss, and the like, for example.

In some embodiments, the consumable according to the present disclosure is a confection. A confection can be a sweet, a lollie, a confectionery, or similar term. The confection may be in the form of any food that is typically perceived to be rich in sugar or is typically sweet. According to particular embodiments of the present disclosure, the confections may be bakery products such as pastries; desserts such as yogurt, jellies, drinkable jellies, puddings. Bavarian cream, blancmange, cakes, brownies, mousse and the like, sweetened food products eaten at tea time or following meals; frozen foods; cold confections, e. g. types of ice cream such as ice cream, ice milk, lacto-ice and the like (food products in which sweeteners and various other types of raw materials are added to milk products, and the resulting mixture is agitated and frozen), and ice confections such as sherbets, dessert ices and the like (food products in which various other types of raw materials are added to a sugary liquid, and the resulting mixture is agitated and frozen), general confections, e. g., baked confections or steamed confections such as crackers, biscuits, buns with bean-jam filling, halvah, alfajor, and the like; rice cakes and snacks; table top products; general sugar confections such as chewing gum (e.g. including compositions which comprise a substantially water-insoluble, chewable gum base, such as chicle or substitutes thereof, including jetulong, guttakay rubber or certain comestible natural synthetic resins or waxes), hard candy, soft candy, mints, nougat candy, jelly beans, fudge, toffee, taffy, Swiss milk tablet, licorice candy, chocolates, gelatin candies, marshmallow, marzipan, divinity, cotton candy, and the like; sauces including fruit flavored sauces, chocolate sauces and the like; edible gels; cremes including butter cremes, flour pastes, whipped cream and the like: jams including strawberry jam, marmalade and the like; and breads including sweet breads and the like or other starch products, and combinations thereof. As referred to herein, “base composition” means any composition which can be a food item and provides a matrix for carrying the sweetener component.

In some embodiments, the present consumable is a condiment that comprises a sweet protein derived from the plant described herein. Condiments, as used herein, are compositions used to enhance or improve the flavor of a food or beverage. Non˜ limiting examples of condiments include ketchup (catsup); mustard: barbecue sauce; butter; chili sauce; chutney; cocktail sauce: curry; dips; fish sauce; horseradish; hot sauce; jellies, jams, marmalades, or preserves, mayonnaise; peanut butter; relish; remoulade; salad dressings (e.g., oil and vinegar, Caesar, French, ranch, bleu cheese, Russian, Thousand Island, Italian, and balsamic vinaigrette), salsa, sauerkraut; soy sauce; steak sauce; syrups; tartar sauce; and Worcestershire sauce.

In some embodiments, the present consumable is a chewing gun that comprises a sweet protein derived from the plant described herein Chewing gum compositions generally comprise a water-soluble portion and a water-insoluble chewable gum base portion. The water soluble portion, which typically includes the sweetener or sweetening composition of the present disclosure, dissipates with a portion of the flavoring agent over a period of time during chewing while the insoluble gum base portion is retained in the mouth. The insoluble gum base generally determines whether a gum is considered chewing gum, bubble gum, or a functional gum.

In some embodiments, the present consumable is a cereal composition that comprises a sweet protein derived from the plant described herein. Cereal compositions typically are eaten either as staple foods or as snacks. Non-limiting examples of cereal compositions for use in particular embodiments include ready-to-eat cereals as well as hot cereals. Ready-to-eat cereals are cereals which may be eaten without further processing (i.e. cooking) by the consumer. Examples of ready-to-eat cereals include breakfast cereals and snack bars. Breakfast cereals typically are processed to produce a shredded, flaky, puffy, or extruded form. Breakfast cereals generally are eaten cold and are often mixed with milk and/or fruit Snack bars include, for example, energy bars, rice cakes, granola bars, and nutritional bars. Hot cereals generally are cooked, usually in either milk or water, before being eaten. Non-limiting examples of hot cereals include grits, porridge, polenta, rice, and rolled oats. Cereal compositions generally comprise at least one cereal ingredient. As used herein, the term “cereal ingredient” denotes materials such as whole or part grains, whole or part seeds, and whole or part grass. Non-limiting examples of cereal ingredients for use in particular embodiments include maize, wheat, rice, barley, bran, bran endosperm, bulgur, soghums, millets, oats, rye, triticale, buchwheat, fonio, quinoa, bean, soybean, amaranth, teff, spelt, and kaniwa.

In some embodiments, the present consumable is a baked good that comprises a sweet protein derived from the plant described herein. Baked goods, as used herein, include ready to eat and all ready to bake products, flours, and mixes requiring preparation before serving. Non-limiting examples of baked goods include cakes, crackers, cookies, brownies, muffins, rolls, bagels, donuts, strudels, pastries, croissants, biscuits, bread, bread products, and buns. Preferred baked goods in accordance with embodiments of the present disclosure can be classified into three groups: bread-type doughs (e.g., white breads, variety breads, soft buns, hard rolls, bagels, pizza dough, and flour tortillas), sweet doughs (e.g., danishes, croissants, crackers, puff pastry, pie crust, biscuits, and cookies), and batters (e.g., cakes such as sponge, pound, devil's food, cheesecake, and layer cake, donuts or other yeast raised cakes, brownies, and muffins) Doughs generally are characterized as being flour-based, whereas batters are more water-based.

In some embodiments, the present consumable is a diary product that comprises a sweet protein derived from the plant described herein. Dairy products and processes for making dairy products suitable for use in the present disclosure are well known to those of ordinary skill in the art. Dairy products, as used herein, comprise milk or foodstuffs produced from milk. Non-limiting examples of dairy products suitable for use in embodiments of the present disclosure include milk, milk cream, sour cream, creme fraiche, buttermilk, cultured buttermilk, milk powder, condensed milk, evaporated milk, butter, cheese, cottage cheese, cream cheese, yogurt, ice cream, frozen custard, frozen yogurt, gelato, via, piima, filmjolk, kajmak, kephir, viili, kumiss, airag, ice milk, casein, ayran, lassi, khoa, or combinations thereof.

In some embodiments, the present consumable is a tabletop flavoring composition that comprises a sweet protein derived from the plant described herein. The tabletop flavoring composition can further include at least one bulking agent, additive, anti-caking agent, functional ingredient or combination thereof. The tabletop flavoring compositions can be packaged in any form known in the art. Non-limiting formis include, but are not limited to, powder form, granular form, packets, tablets, sachets, pellets, cubes, solids, and liquids.

While the forms of plants producing non-native sweet protein and methods of making the same described herein constitute preferred embodiments of this disclosure, it is to be understood that the disclosure is not limited to these precise forms. As will be apparent to those skilled in the art, the various embodiments described above can be combined to provide further embodiments. Aspects of the present transgenic plants, method, and process (including specific components thereof) can be modified, if necessary, to best employ the systems, methods, nodes and components and concepts of the present disclosure. These aspects are considered fully within the scope of the present disclosure as claimed. For example, the various methods described above may omit some acts, include other acts, and/or execute acts in a different order than set out in the illustrated embodiments.

Further, in the transgenic plants and methods of making taught herein, the various acts may be performed in a different order than that illustrated and described. These and other changes can be made to the present systems, methods and articles in light of the above description. In general, in the following claims, the terms used should not be construed to limit the disclosure to the specific embodiments disclosed in the specification and the claims, but should be construed to include all possible embodiments along with the full scope of equivalents to which such claims are entitled. Accordingly, the present disclosure is not limited by the disclosure, but instead its scope is to be determined entirely by the claims in appendix.

### NON-PATENT REFERENCES

Masuda, T., Ueno, Y., and Kitabatake, N. (2005). High yield secretion of the sweet-tasting protein lysozyme from the yeast Pichia pastoris. Protein Expr. Purif. 39, 35-42. Kant, R. (2005). Sweet proteins-potential replacement for artificial low calorie sweeteners. Nutrition Journal 4, 5.
Faus, I. (2000). Recent developments in the characterization and biotechnological production of sweet-tasting proteins. Appl Microbiol Biotechnol 53, 145-151.
Ming, D., and Hellekant, G. (1994). Brazzein, a new high-potency thermostable sweet protein from Pentondiplandra brazzeana B. FEBS Lett 355, 106-108.
Pfeiffer, J. F., Boulton, R. B., and Noble, A. C. (2000). Modeling the sweetness response using time-intensity data. Food Quality and Preference 11, 129-138.
Izawa, H., Ota, M., Kohmura, M., and Ariyoshi, Y. (1996). Synthesis and characterization of the sweet protein brazzein. Biopolymers 39, 95-101.
Assadi-Porter, F. M., Maillet, E. L., Radek, J. T., Quijada, J., Markley, J. L., and Max, M. (2010). Key amino acid residues involved in multi-point binding interactions between brazzein, a sweet protein, and the TIR2-TIR3 human sweet receptor. J Mol Biol 398, 584-599.
Lamphear, B. J., Barker, D. K., Brooks, C. A., Delaney, D. E., Lane, J. R., Beifuss, K., Love, R., Thompson, K., Mayor, J., Clough, R., et al. (2005). Expression of the sweet protein brazzein in maize for production of a new commercial sweetener. Plant Biotechnol J3, 103-114.
Yan S, Song H, Pang D, Zou Q, Li L, et al. (2013) Expression of Plant Sweet Protein Brazzein in the Milk of Transgenic Mice. PLOS ONE 8(10): e76769.
van der Wel, H., Larson, G., Hladik, A., Hladik, C. M., Hellekant, G. Glaser, D. (1989). Isolation and characterization of pentadin, the sweet principle of Pentadiplandra brazzeana Baillon. Chemical Senses, 14(1), 75-79.

All publications, patents and patent applications mentioned in the specification are indicative of the level of those skilled in the art to which this disclosure pertains.

The following examples illustrate preferred, but non-limiting embodiments of the present disclosure.

### EXAMPLES

### Example 1—Production of Watermelon Producing Non-Native Brazzein

Brazzein is a sweet protein originally identified from the Oubli trees (Pentadiplandra brazzeana) found in western Africa. Brazzein is 500 to 2000 times sweeter than sucrose and has the potential to be used as a low-calorie sweetener in the beverage industry. Watermelon represents one of the world's largest fruit production systems by weight and, can be grown in a wide range of geographies. Such favorable economics of watermelon production make the idea of a transgenic watermelon expressing Brazzein very compelling. The present study generated rapid proof of concept dataset and confirmed technical feasibility of producing brazzein in a commercial variety of watermelon.

To design expression cassette sequences for optimization of promoters, signal peptides and codon usage patterns in watermelon. Do novo synthesis and assembly of DNA parts into functional expression vectors. To validate the sequence integrity and design rationale of all expression cassettes by Sanger sequencing

Brazzein proteins can be found in different forms in nature. The minor form, called des-pyrE-bra, which lacks the N-terminal pyroglutamic acid (pyrE) residue, is sweeter than the major form (with pyrE) and therefore selected as the desirable product for this study. The peptide sequence of des-pyrE-bra is set forth in SEQ ID NO: 25 (Ming et al, 1994).

As the peptide sequence of des-pyrE-bra protein does not start with a methionine residue, the following approaches were explored: (1) Addition of a novel start codon (ATG): We expect the novel ATG to produce a protein that varies from the original by a single amino acid. This version is expected to serve as a valuable scientific reagent for rapid testing and optimization of expression systems (designs, promoters and codon usage); (2) Addition of a N-terminal secretion signal: Secretion signal mediated translocation of the protein across the cell membrane results in cleavage of the secretion signal leading to apoplastic accumulation of des-pyrE-bra protein.

To facilitate the expression and detection of these target genes, genetic elements including promoter sequences, epitope tags and terminator sequences were designed for each individual target gene. Four major variables were considered to facilitate the optimal expression: Inclusion of a FLAG tag for detection of brazzein, until anti-brazzein antibody becomes available; Five different signal peptides to facilitate expression, cleavage and secretion of des-pyrE-bra proteins; Six codon usage tables based on codon usage preferences in multiple plant species: Four promoters, including three constitutive promoters previously validated in watermelon, and one fruit specific promoter identified from literature reports.

As shown in Table 1, a total of 18 expression cassettes having different combinations of nucleotide sequences encoding brazzein were constructed. Construction of these expression cassettes was carried out following standard genetic engineering methods. All individual parts needed from this design were synthesized and validated through Sanger sequencing of the plasmids to ensure 100% match to the in silico designs.

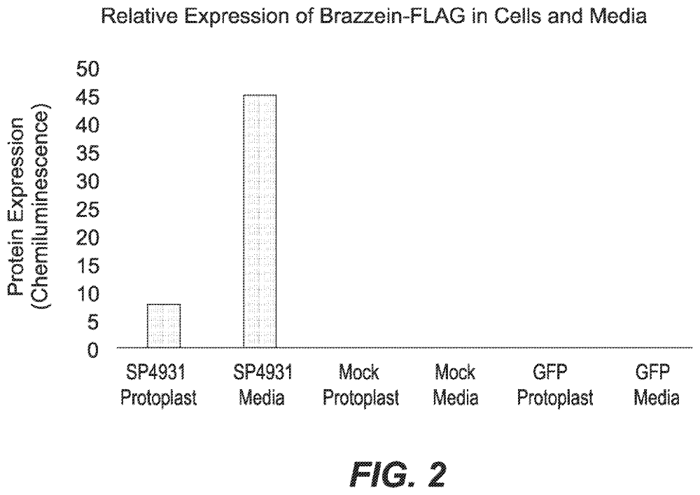

Plasmids housing these expression cassettes were prepared and delivered to watermelon protoplasts isolated from Charleston Gray seedlings. After 24 hours, the protoplasts and the culture media were samples and analyzed using anti-FLAG antibody. As shown in FIG. 2, a FLAG tag containing protein was detected from expression cassette design #4 of Table 1, with a size matching the predicted size of brazzein protein. The results demonstrated the integrity of the cassette design and provide the first evidence that brazzein can be expressed in and then secreted out of watermelon cells.

The following numbered clauses define further example aspects and features of the present disclosure:
1. A plant comprising a genomic transformation event, wherein the genomic transformation event enables the plant to produce a non-native expression or concentration of a sweet protein.
2. The plant of clause 1 being a transgenic plant, wherein the genomic transformation event comprises an expression cassette, wherein the expression cassette comprises one or more of the nucleotide sequences encoding the sweet protein.
3. The plant of clause 2, wherein the expression cassette comprises one or more of the nucleotide sequences as set forth in SEQ ID NOs: 1-24.
4. The plant of any of clauses 2-3, wherein the nucleotide sequences encoding the sweet protein have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-24.
5. The plant of any of the clauses 2-4, wherein the expression cassette comprises one or more regulatory sequences selected from the group consisting of: promoter, spacer, epitope tag, terminator, signal peptide, or combinations thereof.
6. The plant of clause 5, wherein the regulatory sequences have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-6 and 8-13.
7. The plant of any of the clauses 2-6, wherein the expression cassette comprises a promotor operably linked with the nucleotide sequence(s) encoding the sweet protein.
8. The plant of any of the clauses 1-7, wherein the sweet protein is selected from a group consisting of thaumatin, monellin, mabinlin, brazzein, egg white lysozyme, neoculin, pentadin, or a variant thereof, or combinations thereof.
9. The plant of any of clauses 1-8, wherein the sweet protein is brazzein or a variant thereof.
10. The plant of any of clauses 1-9, wherein the sweet protein comprises an amino acid sequence having at least at least 70%, at least 75%, at least 80%, at least 85%, at least 90%, at least 95%, at least 96%, at least 97%, at least 98%, or at least 99% to the amino acid sequence set forth in SEQ ID NO: 25.
11. A plant part obtainable from the plant of any of clauses 1-10, wherein the plant part is derived from organs, tissues, leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures, or any other parts or products of the plant, wherein the plant part comprises the sweet protein.
12. A plant according to any of clauses 1-11, wherein a progeny or an ancestor thereof is a source of the genomic transformation event enabling the progeny and the ancestor to produce the sweet protein.
13. The plant of any of clauses 1-12, wherein the plant is Cucurbitaceae Curcubits.
14. The plant of clause 13, wherein the plant is a watermelon.
15. A sweetener comprising the sweet protein produced by the plant according to any of clauses 1-14.
16. A consumable derived from the plant or a part thereof according to any of clauses 1-14
17. A food, beverage, flavor, or ingredient comprising the sweetener of clause 15.
18. A biosynthetic method for producing a non-native sweet protein, the method comprising:
   (a) combining a plant with a genomic transformation event forming a genetically modified plant, wherein the genomic transformation event enables the genetically modified plant to produce a non-native expression or concentration of the sweet protein,
   (b) growing and regenerating a population of the genetically modified plant;
   (c) selecting the genetically modified plants that produce the sweet protein, and
   (d) harvesting the sweet protein.
19. The method of clause 18 further comprising:
   preparing/providing plasmids comprising an expression cassette, wherein the expression cassette expresses the non-native sweet protein;
   transforming a host cell with the plasmids; and
   transfecting the plant with a plurality of the transformed host cell,
   wherein the genetically modified plant is a transgenic plant.
20. The method of clause 19, wherein the expression cassette comprises a nucleotide sequences encoding the sweet protein.
21. The method of any of clauses 19-20, wherein the expression cassette comprises one or more of the nucleotide sequences as set forth in SEQ ID NOs: 1-24.
22. The method of any of clauses 20-21, wherein the nucleotide sequences have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-24.
23. The method of any of clauses 19-22, wherein the expression cassette comprises one or more regulatory sequences selected from the group consisting of: promoter, spacer, epitope tag, terminator, signal peptide, or combinations thereof.
24. The method of clause 23, wherein the regulatory sequences have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-6 and 8-13.
25. The method of any of clauses 23-24, wherein the expression cassette comprises a promotor operably linked with the nucleotide sequences encoding the sweet protein.
26. The method of any of clauses 18-25, wherein the sweet protein is selected from a group consisting of thaumatin, monellin, mabinlin, brazzein, egg white lysozyme, neoculin, pentadin, or a variant thereof, or combinations thereof.
27. The method of any of clauses 18-26, wherein the sweet protein is brazzein or a variant thereof.
28. The method of any of clauses 18-27, wherein the sweet protein comprises an amino acid sequence having at least at least 70%, at least 75%, at least 80%, at least 85%, at least 90%, at least 95%, at least 96%, at least 97%, at least 98%, or at least 99% to the amino acid sequence set forth in SEQ ID NO: 25.
29. The method of any of clauses 18-28, wherein the plant is Cucurbitaceae Curcubits.
30. The method of clause 29, wherein the plant is a watermelon.
31. A method of making a genetically modified plant producing a non-native sweet protein, comprising combining a plant with a genomic transformation event, wherein the genomic transformation event enables the genetically modified plant to produce a non-native expression or concentration of the sweet protein.
32. The method of clause 31 further comprising:
   preparing/providing plasmids comprising an expression cassette, wherein the expression cassette expresses a non-native sweet protein;
   transforming a host cell with the plasmids; and
   transfecting the plant with a plurality of the transformed host cell,
   wherein the genetically modified plant is a transgenic plant.
33. The method of clause 32, wherein the expression cassette comprises one or more nucleotide sequences encoding the sweet protein.
34. The method of clause 33, wherein the expression cassette comprises one or more of the nucleotide sequences as set forth in SEQ ID NOs: 1-24.
35. The method of any of clauses 33-34, wherein the nucleotide sequences have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-24.
36. The method of any of clauses 32-35, wherein the expression cassette comprises one or more regulatory sequences selected from the group consisting of: promoter, spacer, epitope tag, terminator, signal peptide, or combinations thereof.
37. The method of clause 36, wherein the regulatory sequences have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-6 and 8-13.
38. The method of any of clauses 36-37, wherein the expression cassette comprises a promotor operably linked with the nucleotide sequences encoding the sweet protein.
39. The method of any of clauses 32-38, wherein the sweet protein is selected from a group consisting of thaumatin, monellin, mabinlin, brazzein, egg white lysozyme, neoculin, pentadin, or a variant thereof, or combinations thereof.
40. The method of any of clauses 32-39, wherein the sweet protein is brazzein or a variant thereof.
41. The method of any of clauses 32-40, wherein the sweet protein comprises an amino acid sequence having at least at least 70%, at least 75%, at least 80%, at least 85%, at least 90%, at least 95%, at least 96%, at least 97%, at least 98%, or at least 99% to the amino acid sequence set forth in SEQ ID NO: 25.
42. The method of any of clauses 32-41, wherein the plant is Cucurbitaceae/Curcubits.
43. The method of clause 42, wherein the plant is a watermelon.

The above specification, examples and data provide a complete description of the manufacture and use of the composition of the disclosure. Since many embodiments of the disclosure can be made without departing from the spirit and scope of the disclosure, the disclosure resides in the claims hereinafter appended.

What is claimed is:

1. A plant comprising a genomic transformation event, wherein the genomic transformation event enables the plant to produce a non-native expression or concentration of a sweet protein.

2. The plant of claim 1 being a transgenic plant, wherein the genomic transformation event comprises an expression cassette, wherein the expression cassette comprises one or more of the nucleotide sequences encoding the sweet protein.

3. The plant of claim 2, wherein the expression cassette comprises one or more of the nucleotide sequences as set forth in SEQ ID NOs: 1-24.

4. The plant of any of claims 2-3, wherein the nucleotide sequences encoding the sweet protein have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-24.

5. The plant of any of the claims 2-4, wherein the expression cassette comprises one or more regulatory sequences selected from the group consisting of: promoter, spacer, epitope tag, terminator, signal peptide, or combinations thereof.

6. The plant of claim 5, wherein the regulatory sequences have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-6 and 8-13.

7. The plant of any of the claims 2-6, wherein the expression cassette comprises a promotor operably linked with the nucleotide sequence(s) encoding the sweet protein.

8. The plant of any of the claims 1-7, wherein the sweet protein is selected from a group consisting of thaumatin, monellin, mabinlin, brazzein, egg white lysozyme, neoculin, pentadin, or a variant thereof, or combinations thereof.

9. The plant of any of claims 1-8, wherein the sweet protein is brazzein or a variant thereof.

10. The plant of any of claims 1-9, wherein the sweet protein comprises an amino acid sequence having at least at least 70%, at least 75%, at least 80%, at least 85%, at least 90%, at least 95%, at least 96%, at least 97%, at least 98%, or at least 99% to the amino acid sequence set forth in SEQ ID NO: 25.

11. A plant part obtainable from the plant of any of claims 1-10, wherein the plant part is derived from organs, tissues, leaves, stems, roots, flowers or flower parts, fruits, shoots, gametophytes, sporophytes, pollen, anthers, microspores, egg cells, zygotes, embryos, meristematic regions, callus tissue, seeds, cuttings, cell or tissue cultures, or any other parts or products of the plant, wherein the plant part comprises the sweet protein.

12. A plant according to any of claims 1-11, wherein a progeny or an ancestor thereof is a source of the genomic transformation event enabling the progeny and the ancestor to produce the sweet protein.

13. The plant of any of claims 1-12, wherein the plant is Cucurbitaceae Curcubits.

14. The plant of claim 13, wherein the plant is a watermelon.

15. A sweetener comprising the sweet protein produced by the plant according to any of claims 1-14.

16. A biosynthetic method for producing a non-native sweet protein, the method comprising:
(a) combining a plant with a genomic transformation event forming a genetically modified plant, wherein the genomic transformation event enables the genetically modified plant to produce a non-native expression or concentration of the sweet protein;
(b) growing and regenerating a population of the genetically modified plant;
(c) selecting the genetically modified plants that produce the sweet protein; and
(d) harvesting the sweet protein.

17. The method of claim 16 further comprising:
preparing/providing plasmids comprising an expression cassette, wherein the expression cassette expresses the non-native sweet protein;
transforming a host cell with the plasmids; and
transfecting the plant with a plurality of the transformed host cell,
wherein the genetically modified plant is a transgenic plant.

18. The method of claim 17, wherein the expression cassette comprises a nucleotide sequences encoding the sweet protein.

19. The method of any of claims 17-18, wherein the expression cassette comprises one or more of the nucleotide sequences as set forth in SEQ ID NOs: 1-24.

20. The method of any of claims 18-19, wherein the nucleotide sequences have a sequence identity of at least 70%, at least 75%, at least 80%, at least 85%, at least 90%, at least 95%, at least 96%, at least 97%, at least 98%, or at least 99% to the nucleotide sequences as set forth in SEQ ID NOs: 1-24.
